(19) United States
(12) Patent Application Publication
(10) Pub. No.: US 2026/0144526 A1
(43) Pub. Date: May 28, 2026

(54) ULTRASOUND IMAGING AND THERAPY USING BIAS APERTURES AND ELEMENT CLUSTERS DRIVEN IN QUADRATURE

(71) Applicant: DAXSONICS ULTRASOUND INC., HALIFAX (CA)

(72) Inventors: JEREMY ALASTAIR BROWN, HALIFAX (CA); CHARLES D. EMERY, GILBERT, AZ (US); KATHERINE GWEN LATHAM, DARTMOUTH (CA)

(21) Appl. No.: 19/118,917

(22) PCT Filed: Sep. 15, 2023
(86) PCT No.: PCT/CA2023/051231

§ 371 Date: Apr. 7, 2025

Related U.S. Application Data
(60) Provisional application No. 63/414,368, filed on Oct. 7, 2022.

(51) Int. Cl. A61B 8/00; B06B 1/06; G01S 15/89

(52) U.S. Cl. CPC A61B 8/54; A61B 8/4488; B06B 1/0629; G01S 15/8925; G01S 15/8927; B06B 2201/76

(57) ABSTRACT

Systems and methods are provided for generating and detecting ultrasound energy using an ultrasound array with a reduced set of signal connections. This reduction in signal connections is achieved by employing ultrasound elements that are capable of acoustic transduction under application of a bias, partitioning the array into a set of clusters (subarrays), and delivering time-delayed signals on a per-cluster basis, as opposed to a per-element basis, and employing per-cluster bias apertures, applied in quadrature, to provide the requisite intra-cluster fine phase delays. The signals delivered to the clusters are time-delayed, with each time delay representing an aggregate, cluster-specific coarse delay that can be computed according to a desired transmit phase profile. The per-element fine phase profile, within a given cluster, is generated synthetically or synchronously via the use of two bias apertures that are specific to the cluster and are delivered with respective signals provided in quadrature.

### BACKGROUND

The present disclosure relates to ultrasound therapy and ultrasound imaging.

Systems that can electronically steer and focus an ultrasound beam in three dimensions offer many advantages in both diagnostic imaging and therapy. Unfortunately, these systems also introduce significant challenges that originate at the transducer. This is because it is the design of the transducer that enables electronic steering and focusing of ultrasound in three dimensions. Ultrasound arrays with fine element sampling and independent electronic control in two dimensions, azimuth and elevation, are required. The element sampling in a second dimension when compared a standard linear or phased array increases the number from N (elements in a linear array) to N squared. For example, a 128 element linear array would require 16,384 individual elements in a 2D array. The dramatic increase in elements and the required electrical connections in a concentrated region creates a formidable interconnect issue. In addition to this challenge, the size of a 2D array element is significantly smaller than a conventional linear array element. The size reduction increases the element electrical impedance since it is inversely proportional to element area. The higher electrical impedance causes a reduction in transmit sensitivity and receive signal-to-noise ratio.

### SUMMARY

Systems and methods are provided for generating and detecting ultrasound energy using an ultrasound array with a reduced set of signal connections. This reduction in signal connections is achieved by employing ultrasound elements that are capable of acoustic transduction under application of a bias, partitioning the array into a set of clusters (subarrays), and delivering time-delayed signals on a per-cluster basis, as opposed to a per-element basis, and employing per-cluster bias apertures, applied in quadrature, to provide the requisite intra-cluster fine phase delays. The signals delivered to the clusters are time-delayed, with each time delay representing an aggregate, cluster-specific coarse delay that can be computed according to a desired transmit phase profile. The per-element fine phase profile, within a given cluster, is generated synthetically or synchronously via the use of two bias apertures that are specific to the cluster and are delivered with respective signals provided in quadrature.

Accordingly, in one aspect, there is provided a system for performing ultrasound imaging, the system comprising:
an array of ultrasound transducer elements, each ultrasound element being capable of acoustic transduction when a bias is applied thereto, such that a phase of ultrasound waves emitted therefrom is dependent on a polarity of the bias;
a set of bias conductive paths, each bias conductive path being in electrical communication with a respective bias electrode of an ultrasound element, thereby enabling each ultrasound element to be individually biased;
a set of signal conductive paths, each signal conductive path being configured to deliver a respective signal to a respective subarray of ultrasound elements, thereby enabling the respective signal to be applied to each ultrasound element of the respective subarray of ultrasound elements; and
control and processing circuitry operatively coupled to the set of signal conductive paths and the set of bias conductive paths, the control and processing circuitry comprising at least one processor and associated memory, the memory comprising instructions executable by the processor to perform operations for controlling synthetic transmission of ultrasound energy from the array of ultrasound transducer elements according to a transmit phase aperture, the operations comprising:
   performing a first transmit operation comprising:
      while applying a first transmit bias aperture to the bias conductive paths, delivering a first set of time-delayed transmit signals to the set of signal conductive paths, each transmit signal of the first set of time-delayed transmit signals being delivered to a respective subarray with a respective coarse transmit subarray delay associated with the transmit phase aperture;
   performing a second transmit operation comprising:
      while applying a second transmit bias aperture to the bias conductive paths, delivering a second set of time-delayed transmit signals to the signal conductive paths, the second set of time-delayed transmit signals being generated in quadrature relative to the first set of time-delayed transmit signals;
wherein the first transmit bias aperture and the second transmit bias aperture are configured such that when the first transmit operation and the second transmit operation are performed, a fine phase delay associated with the transmit phase aperture is synthetically generated for each transducer element, such that a combination of the per-subarray coarse transmit subarray delays and the per-element fine phase delays synthetically generate or approximate the transmit phase aperture.

In some example implementations of the system, the control and processing circuitry is further configured such that for at least one subarray, the coarse transmit subarray delay associated with the subarray is a statistical measure generated based on processing a set of per-element time delays, within the subarray that would be needed to generate the transmit phase aperture according to a single transmit operation.

In some example implementations of the system, In some example implementations of the system, the control and processing circuitry is further configured such that for at least one subarray, the coarse transmit subarray delay associated with the subarray is determined based on a relative geometrical location of the subarray within the array of ultrasound transducer elements.

In some example implementations of the system, the control and processing circuitry is configured such that bias levels of the first transmit bias aperture and the second transmit bias aperture are generated according to a discrete set of bias levels, the discrete set of bias levels comprising at least three distinct bias levels.

In some example implementations of the system, the control and processing circuitry is configured such that the bias levels of the first transmit bias aperture and the bias levels of the second transmit bias aperture are obtained from a lookup table, the lookup table associating, for each phase range of a plurality of phase ranges, a suitable first transmit aperture bias value selected from the selected from the discrete set of bias levels, such that a synthetic transmit aperture generated by compounding of the first transmit operation and the second transmit operation approximates the transmit phase aperture.

In some example implementations of the system, the control and processing circuitry is further configured such that for at least one subarray, the first transmit operation is performed as a set of first synthetic transmit operations and the second transmit operation is performed as a corresponding set of second synthetic transmit operations, and wherein the set of first synthetic transmit operations and the set of second synthetic transmit operations are configured to reduce or avoid phase wrapping within the subarray.

In some example implementations of the system, the control and processing circuitry is further configured such that when performing a given first synthetic transmit operation associated with the first transmit operation, a respective subaperture of the elements of the subarray is biased according to the first transmit bias aperture with a remainder of the elements of the subarray being unbiased, and wherein the given first synthetic transmit operation has a corresponding second synthetic transmit operation associated with the second transmit operation, in which the subaperture of the electrodes of the subarray is biased according to the second transmit bias aperture with the remainder of the elements of the subarray being unbiased, and wherein the given first synthetic transmit operation and the corresponding second synthetic transmit operation are performed using a coarse transmit subaperture delay selected to reduce or avoid phase wrapping within the subaperture of elements of the subarray.

In some example implementations of the system, the control and processing circuitry is further configured such that the coarse transmit subaperture delay is a statistical measure generated based on processing a set of per-element time delays, within the subaperture of the subarray, that would be needed to generate the transmit phase aperture according to a single transmit operation.

In some example implementations of the system, the control and processing circuitry is further configured such that the number of synthetic transmit operations associated with a given subarray is dependent on a focal location associated with the transmit phase aperture.

In some example implementations of the system, the control and processing circuitry is further configured such that the number of synthetic transmit operations associated with a given subarray is selected to minimize a variation in a signal-to-noise ratio among synthetic transmit operations associated with the given subarray.

In some example implementations of the system, two or more of the subarrays have different sizes.

In some example implementations of the system, a central subarray has a larger size than a peripheral subarray.

In some example implementations of the system, at least on subarray is sufficiently small to avoid phase wrapping within a pre-selected steering range.

In some example implementations of the system, the control and processing circuitry is configured such that the transmit phase aperture associated with a real focus, such that the first transmit operation and the second transmit operation synthetically focus ultrasound energy at the real focus.

In some example implementations of the system, the real focus is a first real focus, and wherein the control and processing circuitry is configured such that an additional first transmit operation and an additional second transmit operation are performed to synthetically focus ultrasound energy at a second real focus residing proximal to the first real focus, wherein the additional first transmit operation is performed using the first transmit bias aperture and the additional second transmit operation is performed using the second transmit bias aperture, such that the second real focus is obtained by modifying the coarse transmit subarray delays applied to the transmit signals without modifying the first transmit bias aperture and the second transmit bias aperture.

In some example implementations of the system, the control and processing circuitry is configured to perform additional first and second transmit operations to synthetically focus ultrasound energy at a plurality of focal locations within a selected sector by modifying the coarse transmit subarray delays applied to the transmit signals without modifying the first transmit bias aperture and the second transmit bias aperture.

In some example implementations of the system, the control and processing circuitry is configured such that the transmit phase aperture is associated with a virtual focus, such that the first transmit operation and the second transmit operation synthetically generate ultrasound energy according to the virtual focus.

In some example implementations of the system, the control and processing circuitry is further configured to perform additional operations for synthetically receiving ultrasound energy according to a receive phase aperture, comprising:
in response to the first transmit operation, performing a first receive operation by receiving a first set of receive signals while applying a first receive bias aperture;
in response to the second transmit operation, performing a second receive operation by receiving a second set of receive signals while applying the first receive bias aperture;
performing a third transmit operation by repeating the first transmit operation, and in response to the third transmit operation, performing a third receive operation by receiving a third set of receive signals while applying a second receive bias aperture and applying a quarter-wave time delay to the third set of receive signals; performing a fourth transmit operation by repeating the second transmit operation, and in response to the fourth transmit operation, performing a fourth receive operation by receiving a fourth set of receive signals while applying the second receive bias aperture and applying a quarter-wave time delay to the fourth set of receive signals;
wherein the first receive bias aperture and the second receive bias aperture are configured to synthetically generate a fine phase delay associated with the receive phase aperture;
beamforming each of the first set of receive signals, the second set of receive signals, the third set of receive signals and the fourth set of receive signals, according to coarse receive subaperture delays associated with the receive phase aperture, and summing the resulting first beamformed receive signal, second beamformed receive signal, third beamformed receive signal, a fourth beamformed receive signal to obtain a final receive beamformed signal.
19. The system according to claim 18 wherein the control and processing circuitry is configured such that bias levels of the first receive bias aperture and the second receive bias aperture are generated according to a discrete set of bias levels, the discrete set of bias levels comprising at least three distinct bias levels.

In some example implementations of the system, the control and processing circuitry is configured such that bias levels of the first receive bias aperture and the bias levels of the second receive bias aperture are obtained from a lookup table, the lookup table associating, for each phase range of a plurality of phase ranges, a suitable first receive aperture bias value selected from the discrete set of bias levels and a suitable second receive aperture bias level selected from the discrete set of bias levels, such that a synthetic receive aperture generated by the compounding of the receive operations approximates the receive phase aperture.

In some example implementations of the system, the control and processing circuitry is configured such that the set of synthetic transmit and receive operations are performed in a sequence that minimizes switching between the bias apertures.

In some example implementations of the system, the first receive bias aperture is the same as the first transmit bias aperture and the second receive bias aperture is the same as the second transmit bias aperture.

In some example implementations of the system, the first transmit operation, the second transmit operation, the third transmit operation, and the fourth transmit operation are a first set of synthetic transmit operations and wherein the first receive operation, the second receive operation, the third receive operation, and the fourth receive operation are a first set of synthetic receive operations, and wherein the control and processing circuitry is configured to perform at least one additional set of synthetic transmit operations and at least one additional set of synthetic receive operations, wherein each set of synthetic transmit operations is configured to synthetically generate an ultrasound field that approximates a plane wave, and wherein the plane waves associated with the sets of synthetic transmit operations spatially overlap within a region;
wherein each set of synthetic receive operations is configured to synthetically focus ultrasound energy from a different location within the region.

In some example implementations of the system, the control and processing circuitry is configured such that each set of synthetic receive operations synthetically focuses ultrasound energy from a different location by modifying the coarse receive subarray delays in the absence of modifying the first receive bias aperture and the second receive bias aperture.

In some example implementations of the system, the control and processing circuitry is configured such that each set of synthetic receive operations synthetically focuses ultrasound energy from a different location, at least in part, by modifying the coarse receive subarray delays, and wherein at least two different pairs of the first receive bias aperture and the second receive bias aperture are employed when performing the sets of synthetic receive operations.

In some example implementations of the system, the array of ultrasound transducer elements comprises an electrostrictive material.

In some example implementations of the system, the array of ultrasound transducer elements is formed from an array of capacitive micromachined ultrasound transducer elements.

In another aspect, there is provided a method of performing ultrasound imaging, the method comprising:
providing an ultrasound device comprising:
   an array of ultrasound transducer elements, each ultrasound element being capable of acoustic transduction when a bias is applied thereto, such that a phase of ultrasound waves emitted therefrom is dependent on a polarity of the bias;
   a set of bias conductive paths, each bias conductive path being in electrical communication with a respective bias electrode of an ultrasound element, thereby enabling each ultrasound element to be individually biased; and
   a set of signal conductive paths, each signal conductive path being configured to deliver a respective signal to a respective subarray of ultrasound elements, thereby enabling the respective signal to be applied to each ultrasound element of the respective subarray of ultrasound elements; and
performing a first transmit operation comprising:
   while applying a first transmit bias aperture to the bias conductive paths, delivering a first set of time-delayed transmit signals to the set of signal conductive paths, each transmit signal of the first set of time-delayed transmit signals being delivered to a respective subarray with a respective coarse transmit subarray delay associated with a transmit phase aperture;
performing a second transmit operation comprising:
   while applying a second transmit bias aperture to the bias conductive paths, delivering a second set of time-delayed transmit signals to the signal conductive paths, the second set of time-delayed transmit signals being generated in quadrature relative to the first set of time-delayed transmit signals;
wherein the first transmit bias aperture and the second transmit bias aperture are configured such that when the first transmit operation and the second transmit operation are performed, a fine phase delay associated with the transmit phase aperture is synthetically generated for each transducer element, such that a combination of the per-subarray coarse transmit subarray delays and the per-element fine phase delays synthetically generate or approximate the transmit phase aperture.

In another aspect, there is provided a system for delivering ultrasound energy, the system comprising:
an array of ultrasound transducer elements, each ultrasound element comprising a first sub-element and a second sub-element residing adjacent to one another, each sub-element being capable of acoustic transduction when a bias is applied thereto, such that a phase of ultrasound waves emitted therefrom is dependent on a polarity of the bias;
a first set of bias conductive paths, each first bias conductive path being in electrical communication with a respective bias electrode of a first sub-element, thereby enabling each first sub-element to be individually biased;
a second set of bias conductive paths, each second bias conductive path being in electrical communication with a respective bias electrode of a second sub-element, thereby enabling each second sub-element to be individually biased;
a first set of signal conductive paths, each first signal conductive path being configured to deliver a respective signal to a respective set of first sub-elements of a subarray of ultrasound elements, thereby enabling the respective signal to be applied to each first sub-element of the respective subarray of ultrasound elements;
a second set of signal conductive paths, each second signal conductive path being configured to deliver a respective signal to a respective set of second sub-elements of a subarray of ultrasound elements, thereby enabling the respective signal to be applied to each second sub-element of the respective subarray of ultrasound elements; and
control and processing circuitry operatively coupled to the set of signal conductive paths, the first set of bias conductive paths and the second set of bias conductive paths, the control and processing circuitry comprising at least one processor and associated memory, the memory comprising instructions executable by the processor to perform operations for controlling transmission of ultrasound energy from the array of ultrasound transducer elements according to a transmit phase aperture, the operations comprising:
   simultaneously performing a first transmit operation and a second transmit operation;
   the first transmit operation comprising:
      while applying a first transmit bias aperture to the first set of bias conductive paths, delivering a first set of time-delayed transmit signals to the first set of signal conductive paths, each transmit signal of the first set of time-delayed transmit signals being delivered to a respective subarray with a respective coarse transmit subarray delay associated with the transmit phase aperture;
   the second transmit operation comprising:
      while applying a second transmit bias aperture to the second bias conductive paths, delivering a second set of time-delayed transmit signals to the second set of signal conductive paths, the second set of time-delayed transmit signals being generated in quadrature relative to the first set of time-delayed transmit signals;
the first transmit bias aperture and the second transmit bias aperture being configured such that when the first transmit operation and the second transmit operation are simultaneously performed, each pair of first and second sub-elements generates a net fine phase delay associated with the transmit phase aperture, such that a combination of the per-subarray coarse transmit subarray delays and the per-sub-element fine phase delays generate or approximate the transmit phase aperture.

In some example implementations of the system, the control and processing circuitry is further configured such that for at least one subarray, the coarse transmit subarray delay associated with the subarray is a statistical measure generated based on processing a set of per-element time delays, within the subarray that would be needed to generate the transmit phase aperture according to a single transmit operation.

In some example implementations of the system, the control and processing circuitry is further configured such that for at least one subarray, the coarse transmit subarray delay associated with the subarray is determined based on a relative geometrical location of the subarray within the array of ultrasound transducer elements.

In some example implementations of the system, the control and processing circuitry is configured such that bias levels of the first transmit bias aperture and the second transmit bias aperture are generated according to a discrete set of bias levels, the discrete set of bias levels comprising at least three distinct bias levels.

In some example implementations of the system, the control and processing circuitry is configured such that the bias levels of the first transmit bias aperture and the bias levels of the second transmit bias aperture are obtained from a lookup table, the lookup table associating, for each phase range of a plurality of phase ranges, a suitable first transmit aperture bias value selected from the selected from the discrete set of bias levels, such that the first transmit operation and the second transmit operation approximates the transmit phase aperture.

In some example implementations of the system, two or more of the subarrays have different sizes.

In some example implementations of the system, a central subarray has a larger size than a peripheral subarray.

In some example implementations of the system, at least on subarray is sufficiently small to avoid phase wrapping within a pre-selected steering range.

In some example implementations of the system, the control and processing circuitry is configured such that the transmit phase aperture associated with a real focus, such that the first transmit operation and the second transmit operation focus ultrasound energy at the real focus.

In some example implementations of the system, the real focus is a first real focus, and wherein the control and processing circuitry is configured such that an additional first transmit operation and an additional second transmit operation are performed to focus ultrasound energy at a second real focus residing proximal to the first real focus, wherein the additional first transmit operation is performed using the first transmit bias aperture and the additional second transmit operation is performed using the second transmit bias aperture, such that the second real focus is obtained by modifying the coarse transmit subarray delays applied to the transmit signals without modifying the first transmit bias aperture and the second transmit bias aperture.

In some example implementations of the system, the control and processing circuitry is configured to perform additional first and second transmit operations to focus ultrasound energy at a plurality of focal locations within a selected sector by modifying the coarse transmit subarray delays applied to the transmit signals without modifying the first transmit bias aperture and the second transmit bias aperture.

In some example implementations of the system, the control and processing circuitry is configured such that the transmit phase aperture is associated with a virtual focus, such that the first transmit operation and the second transmit operation generate ultrasound energy according to the virtual focus.

In some example implementations of the system, the control and processing circuitry is further configured to perform additional operations for receiving ultrasound energy according to a receive phase aperture, comprising:
in response to the first transmit operation and the second transmit operation:
   performing a first receive operation by receiving a first set of receive signals from the first set of signal conductive paths while applying a first receive bias aperture to the first set of bias conductive paths; and
   performing a second receive operation by receiving a second set of receive signals from the second set of signal conductive paths while applying a second receive bias aperture and applying a quarter-wave time delay to the second set of receive signals;
simultaneously performing a third transmit operation and a fourth transmit operation by repeating the first transmit operation and the second transmit operation;
in response to the third transmit operation and the fourth transmit operation:
   performing a third receive operation by receiving a third set of receive signals from the first set of signal conductive paths while applying the second receive bias aperture to the first set of bias conductive paths and applying a quarter-wave time delay to the second set of receive signals; and
   performing a fourth receive operation by receiving a fourth set of receive signals from the second set of signal conductive paths while applying the first receive bias aperture;
wherein the first receive bias aperture and the second receive bias aperture are configured to synthetically generate a fine phase delay associated with the receive phase aperture;
beamforming each of the first set of receive signals, the second set of receive signals, the third set of receive signals and the fourth set of receive signals, according to coarse receive subaperture delays associated with the receive phase aperture, and summing the resulting first beamformed receive signal, second beamformed receive signal, third beamformed receive signal, a fourth beamformed receive signal to obtain a final receive beamformed signal.

In some example implementations of the system, the control and processing circuitry is configured such that bias levels of the first receive bias aperture and the second receive bias aperture are generated according to a discrete set of bias levels, the discrete set of bias levels comprising at least three distinct bias levels.

In some example implementations of the system, the control and processing circuitry is configured such that bias levels of the first receive bias aperture and the bias levels of the second receive bias aperture are obtained from a lookup table, the lookup table associating, for each phase range of a plurality of phase ranges, a suitable first receive aperture bias value selected from the discrete set of bias levels and a suitable second receive aperture bias level selected from the discrete set of bias levels, such that the receive operations approximate the receive phase aperture.

In some example implementations of the system, the first receive bias aperture is the same as the first transmit bias aperture and the second receive bias aperture is the same as the second transmit bias aperture.

In some example implementations of the system, the first transmit operation, the second transmit operation, the third transmit operation, and the fourth transmit operation are a first set of transmit operations and wherein the first receive operation, the second receive operation, the third receive operation, and the fourth receive operation are a first set of receive operations, and wherein the control and processing circuitry is configured to perform at least one additional set of transmit operations and at least one additional set of receive operations, wherein each set of transmit operations is configured to generate an ultrasound field that approximates a plane wave, and wherein the plane waves associated with the sets of synthetic transmit operations spatially overlap within a region;
wherein each set of receive operations is configured to focus ultrasound energy from a different location within the region.

In some example implementations of the system, the control and processing circuitry is configured such that each set of receive operations focuses ultrasound energy from a different location by modifying the coarse receive subarray delays in the absence of modifying the first receive bias aperture and the second receive bias aperture.

In some example implementations of the system, the control and processing circuitry is configured such that each set of receive operations focuses ultrasound energy from a different location, at least in part, by modifying the coarse receive subarray delays, and wherein at least two different pairs of the first receive bias aperture and the second receive bias aperture are employed when performing the sets of receive operations.

In some example implementations of the system, the array of ultrasound transducer elements comprises an electrostrictive material.

In some example implementations of the system, the array of ultrasound transducer elements is formed from an array of capacitive micromachined ultrasound transducer elements.

In another aspect, there is provided a method for delivering ultrasound energy, the system comprising:
providing an ultrasound device comprising:
   an array of ultrasound transducer elements, each ultrasound element comprising a first sub-element and a second sub-element residing adjacent to one another, each sub-element being capable of acoustic transduction when a bias is applied thereto, such that a phase of ultrasound waves emitted therefrom is dependent on a polarity of the bias;
   a first set of bias conductive paths, each first bias conductive path being in electrical communication with a respective bias electrode of a first sub-element, thereby enabling each first sub-element to be individually biased;
   a second set of bias conductive paths, each second bias conductive path being in electrical communication with a respective bias electrode of a second sub-element, thereby enabling each second sub-element to be individually biased;
   a first set of signal conductive paths, each first signal conductive path being configured to deliver a respective signal to a respective set of first sub-elements of a subarray of ultrasound elements, thereby enabling the respective signal to be applied to each first sub-element of the respective subarray of ultrasound elements; and
   a second set of signal conductive paths, each second signal conductive path being configured to deliver a respective signal to a respective set of second sub-elements of a subarray of ultrasound elements, thereby enabling the respective signal to be applied to each second sub-element of the respective subarray of ultrasound elements; and
simultaneously performing a first transmit operation and a second transmit operation;
the first transmit operation comprising:
   while applying a first transmit bias aperture to the first set of bias conductive paths, delivering a first set of time-delayed transmit signals to the first set of signal conductive paths, each transmit signal of the first set of time-delayed transmit signals being delivered to a respective subarray with a respective coarse transmit subarray delay associated with a transmit phase aperture;
the second transmit operation comprising:
   while applying a second transmit bias aperture to the second bias conductive paths, delivering a second set of time-delayed transmit signals to the second set of signal conductive paths, the second set of time-delayed transmit signals being generated in quadrature relative to the first set of time-delayed transmit signals;
the first transmit bias aperture and the second transmit bias aperture being configured such that when the first transmit operation and the second transmit operation are simultaneously performed, each pair of first and second sub-elements generates a net fine phase delay associated with the transmit phase aperture, such that a combination of the per-subarray coarse transmit subarray delays and the per-sub-element fine phase delays generate or approximate the transmit phase aperture.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 shows an overhead view of a convention 2D ultrasound array.

FIG. 2 illustrates an example an ultrasound array that has been partitioned into a set of clusters.

FIG. 3 shows an example 16×16 array that has been partitioned into 4×4 clusters.

FIG. 4 shows the reduced set of signal channels needed to interface with the clusters of FIG. 3.

FIG. 5 shows signal delivery to a single cluster.

FIG. 6 schematically shows the per-element bias connections provided within a single example cluster.

FIG. 7A schematically shows the ability to engineering a specific phase for every element of the cluster.

FIG. 7B is a table showing possible effective phases obtainable using two bias values.

FIG. 7C is a table showing possible effective phases when electrostrictive apodization in applied.

FIG. 7D is a table showing assigned bias amplitudes for five bias values based on the ideal phase.

FIG. 7E is a table showing assigned bias amplitudes for seven bias values based on the ideal phase.

FIG. 7F and FIG. 7G demonstrate different methods of applying signals in quadrature.

FIG. 8A shows the remaining distance from each element within the cluster to the intended focus (in millimeters) for an example cluster.

FIG. 8B shows the labels for the elements within the cluster shown in FIG. 8A, numbered from 1 to 64.

FIG. 8C plots the distances corresponding to the elements shown in FIG. 8B.

FIG. 8D sorts the distances shown in FIG. 8C from high to low.

FIG. 8E plots the phase corresponding to the distances shown in FIG. 8D.

FIG. 8F groups the elements with regard to phase traversal of the unit circle.

FIGS. 9A and 9B show the number of wraps around the unit circle for a cluster near the center of the array x=0.3 mm, y=0.3 mm and one at the corner x=−6.9 mm, y=−6.9 mm.

FIGS. 9C and 9D show results for when the beam is steered 30 degrees to x=0 mm, y=35.0 mm, z=60.6 mm where the total focal length remains 70 mm, the center cluster (FIG. 9C) as well as the corner of the array (FIG. 9D) show no phase wrap.

FIGS. 9E and 9F show results for when the beam is steered 45 degrees in theta and phi, where both the cluster near the center of the array as well as the one near the opposite edge of the focus show at least one phase wrap.

FIGS. 9G and 9H show results for when the focus initially resides at x=0 mm, y=0 mm, z=70 mm, showing the number of wraps around the unit circle for a cluster near the center of the array x=0.3 mm, y=0.3 mm and one at the corner x=−6.9 mm, y=−6.9 mm.

FIGS. 9I and 9J show results for when the beam is steered 30 degrees to x=0 mm, y=35.0 mm, z=60.6 mm where the total focal length remains 70 mm, for the center cluster (FIG. 9I) as well as the corner of the array (FIG. 9J).

FIGS. 9K and 9L show results for when the beam is steered 45 degrees in theta and phi, where both the cluster near the center of the array as well as the one near the opposite edge of the focus show at multiple phase wraps.

FIG. 10 illustrates different example shapes of clusters and cluster elements.

FIGS. 11A, 11B and 11C illustrate synthetic imaging, with FIG. 11C showing example combinations of bias values that result in net phases for constructing the cosine and sine bias apertures to achieve a desired net synthetic phase profile.

FIGS. 12A, 12B, 12C and 12D show the timing sequence of the four transmit and receive events employed during synthetic quadrature excitation.

FIG. 12E is a table showing an example sequence for performing four pulse-echo events.

FIG. 13 shows a 2D array, along with a potential 3D volume FOV based on multiple transmit-receive vectors.

FIGS. 14A, 14B and 14C show PSF results based on the imaging scheme illustrated in FIG. 13.

FIG. 15A shows the resulting one-way response for the straight ahead focus which combines the responses using the straight ahead focus and the slightly steered focus (4 degrees).

FIG. 15B shows results obtained using a steered focus.

FIG. 15C shows a response for beam steered 4 degrees if only the ideal bias patterns are used.

FIGS. 16 and 17 illustrates example embodiment in which the first and second quadrature transmit operations may be performed simultaneously for generating a real transmit focus, as opposed to a synthetic transmit focus.

FIG. 18 illustrates an example system for performing ultrasound imaging and therapy using bias apertures and element clusters driven in quadrature.

FIGS. 19A and 19B shows the resulting one-way PSF of the 2D array for two different focal conditions for a gold standard fully sampled 2D array: Focus1, x=0 mm, 17.3 mm, 30.0 mm (theta=30 degrees, phi=0 degrees); FIG. 19A, Focus2, x=8.65 mm, 14.98 mm, 30 mm (theta=30 degrees, phi=30 degrees); FIG. 19B.

FIGS. 20A and 20B show the pulse at the intended focus for two simulations noted above.

FIGS. 21A and 21B shows the one-way PSF for the same two foci captured in FIGS. 19A and 19B for the case of a quad cluster array.

FIGS. 22A and 22B and show the transmit pulse at the focus for the simulated quad cluster array.

FIGS. 23A and 23B show the one-way point spread functions (PSFs) for the two different steering angles for a second simulated quad cluster array.

FIGS. 24A and 24B show the pulse at the transmit focus for the second simulated quad cluster array.

FIGS. 25A and 25B show the point spread function for two different steering angles for a third simulated quad cluster array.

FIGS. 26A and 26B show the pulse at the transmit focus for the third simulated quad cluster array.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

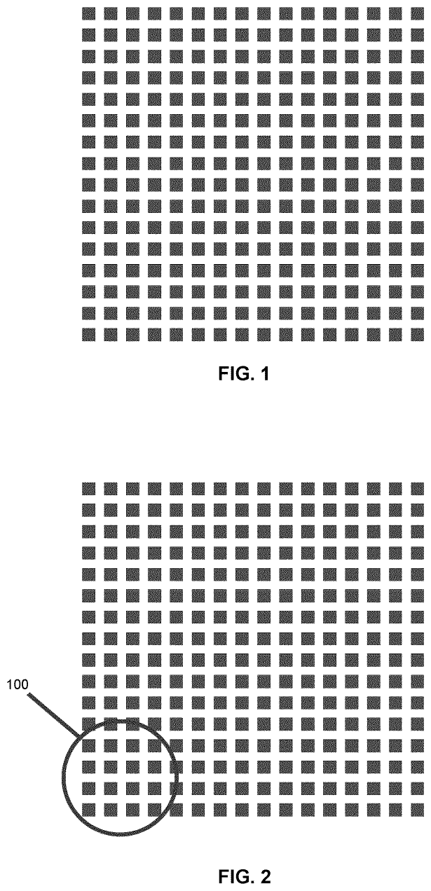

FIG. 1 shows an overhead view of a convention 2D ultrasound array, showing an example configuration that employs 256 elements in a 16×16 configuration. Conventionally, this type of array would require 256 separate transmit and receive channels that offer independent control of excitations and time delays in order to generate a desired phase aperture across the array (e.g. for transmitting a focused or defocused ultrasound pulse).

In contrast to the conventional 2D ultrasound array shown in FIG. 1, the present inventors have discovered that it is possible to synthetically generate a desired transmit phase profile across an ultrasound array using far fewer signal connections, provided that the ultrasound array elements are capable of acoustic transduction under application of a bias, such that a phase of emitted ultrasound waves is dependent on a polarity of the bias. This reduction in signal connections can be achieved, as will be shown below, by partitioning the array of elements into a set of clusters (subarrays) and delivering the signals on a per-cluster basis, as opposed to a per-element basis, thereby requiring fewer signal connections than a conventional ultrasound array, and employing per-cluster bias apertures, applied synthetically in quadrature, to provide the requisite intra-cluster fine phase delays.

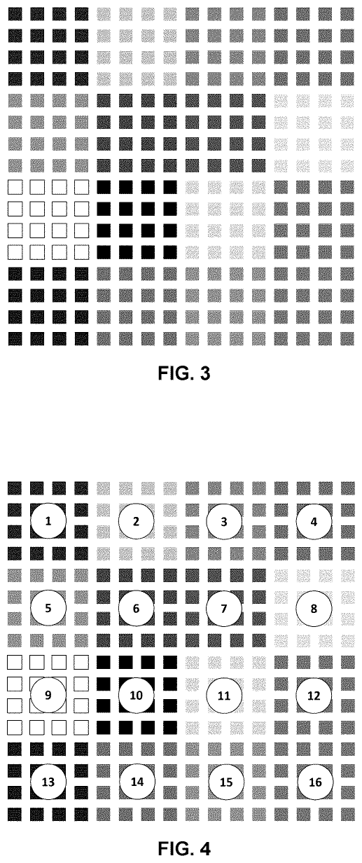

FIG. 2 illustrates an example an ultrasound array that has been partitioned into a set of clusters. In the present example case, a cluster 100 is shown as a group of 16 elements and has a size of 4 by 4. Likewise, FIG. 3 shows an example 16×16 array that has been partitioned into 4×4 clusters according to the present methods. The individual cluster of elements have been identified by different shades of gray. In this case, every cluster is a 4 by 4 grouping of elements, such that the array consists of 16 distinct clusters. As can be seen in FIG. 4, which shows the reduced number of signal channels needed, an array that would have been conventionally addressed by 256 system channels has been simplified to an array that only requires 16 signal channels and 256 bias circuits, a configuration that is more cost effective and simpler to deploy.

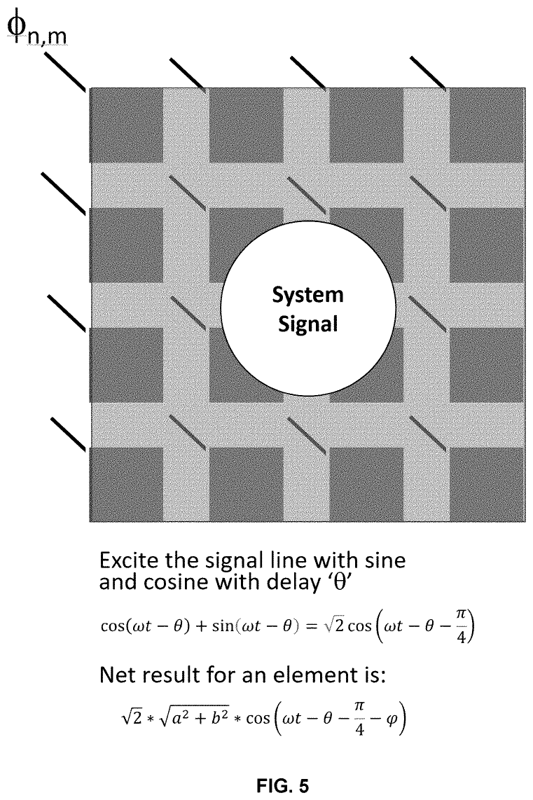

The signals that are delivered to the clusters are respectively delayed by respective beamforming time delays, with each time delay representing an aggregate, cluster-specific coarse delay that can be computed according to a desired transmit phase profile. A time-delayed signal is delivered to a given cluster such that the signal is provided to all elements of the given cluster, through a signal conductive path that is defined on one side of the array. A signal conductive path associated with a given cluster is brought into electrical communication with one or more electrodes that are associated with the cluster such that the signal is applied to the entire cluster. An example of the delivery of a signal to a cluster is illustrated in FIG. 5.

The coarse time delay that is employed to delay the signal applied to a given cluster may be determined several different ways, such as via a statistical measure generated based on processing a set of per-element time delays, within the cluster, that would be needed to generate the transmit phase aperture according to a single transmit operation. According to one example method, the coarse delay is the mean of the time delays of all the element delays within the cluster. The time delay could also be the median of the time delays across the elements. The time delay may also be dependent on the effective geometric location of the cluster.

The signals that are delivered to the clusters only provide the coarse time delays associated with the desired phase profile and do not provide the required per-element fine phasing that is necessary to generate or approximate the desired transmit phase profile across the ultrasound array. The per-element fine phase profile, within a given cluster, is generated synthetically via the use of two bias apertures that are specific to the cluster, where a first bias aperture is applied to the cluster when delivering the signal to the cluster according to a first transmit operation (with the appropriate coarse time delay for the cluster), and a second bias aperture is applied to the cluster when delivering a quadrature version of the signal (with the same appropriate coarse time delay for the cluster) according to a second transmit operation. As will be shown below, by judiciously selecting the two bias apertures that are synthetically applied in quadrature, the fine phase delay associate with the desired transmit phase profile can be recovered or approximated. As a result, when the suitable bias apertures are applied to the clusters of the array during the two transmit operations, and when the signals delivered to the clusters are time-delayed by the suitable coarse time delays, the desired transmit phase aperture can be synthetically generated or approximated.

As explained above, the signal channel that is delivered to each cluster provides the coarse ‘macro-delay’ and the bias lines that address each element of a given cluster provide the ‘micro-delays’ via the synthetic transmit operations, with the combination of the macro and micro delays synthetically generating the desired transmit phase profile within the cluster. This may also be explained as coarse delay control (system channels) and fine delay control (bias connections). According to the present example embodiment, the delays that are provided to the signals are time-based, whereas the delays from the bias connections are phase-based and dependent on the operational frequency or center frequency of the waveform at the intended focus. The net delay at an element is the combination of the macro-delay from the system console and the phase delay generated via a bias control.

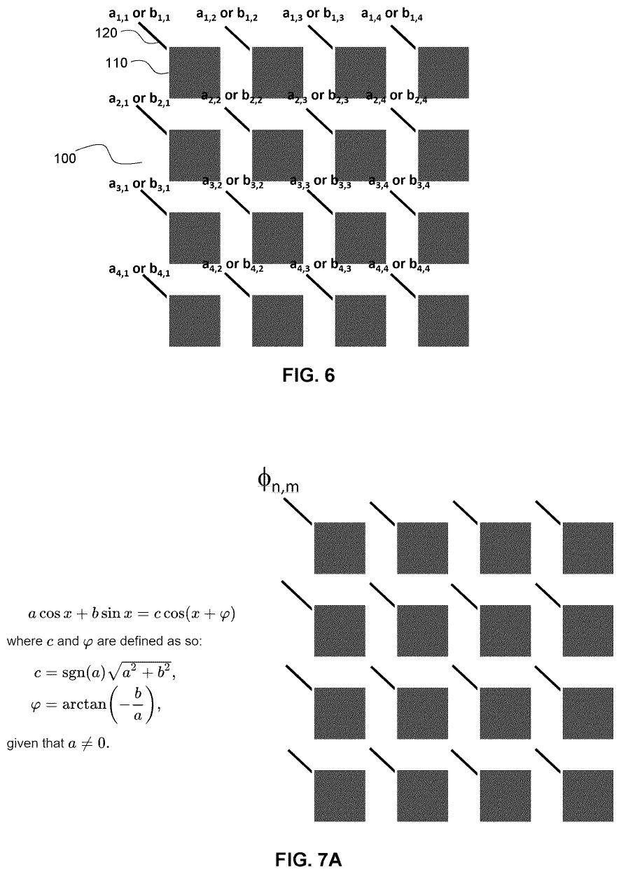

FIG. 6 schematically shows the per-element bias connections provided within a single example cluster (an example 16-element cluster from FIG. 2). As shown in the figure, each individual element 110 has an independent electrical connection 120 to apply a DC bias (i.e. an individual bias conductive path), and each element is capable of acoustic transduction under application of a respective bias, such that a phase of emitted ultrasound waves is dependent on a polarity of the bias. FIG. 6 shows that each element 100 has a bias electrode connected to a respective unique respective bias line 120.

Each element 100 in the example cluster shown in FIG. 6 is shown identified by the subscripts m,n. The figure shows that two different bias apertures, a_{m,n} and b_{m,n}, can be applied to the cluster. As will be described in further detail below, the individual per-element values of these two bias apertures can be selected such that when the bias apertures are sequentially applied, respectively, with signals delivered in quadrature, the net synthetic phase associated with each element within the cluster can be uniquely controlled. In other words, by delivering the combination of (i) a signal with bias aperture a_{m,n} and (i) a quadrature version of the signal with bias aperture b_{m,n}, the individual amplitudes a_{m,n} and b_{m,n} may be defined with suitable amplitudes and signs to achieve a net synthetic phase profile that equals or approximates a desired local phase profile across the cluster.

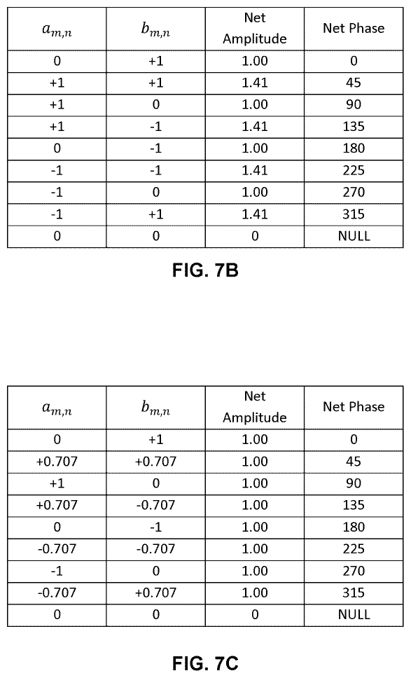

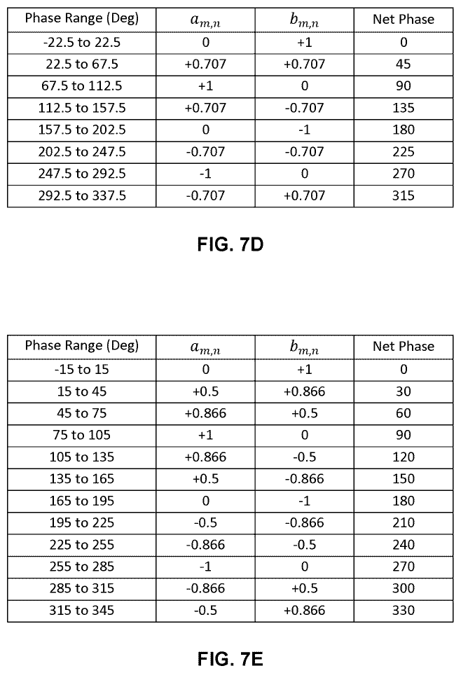

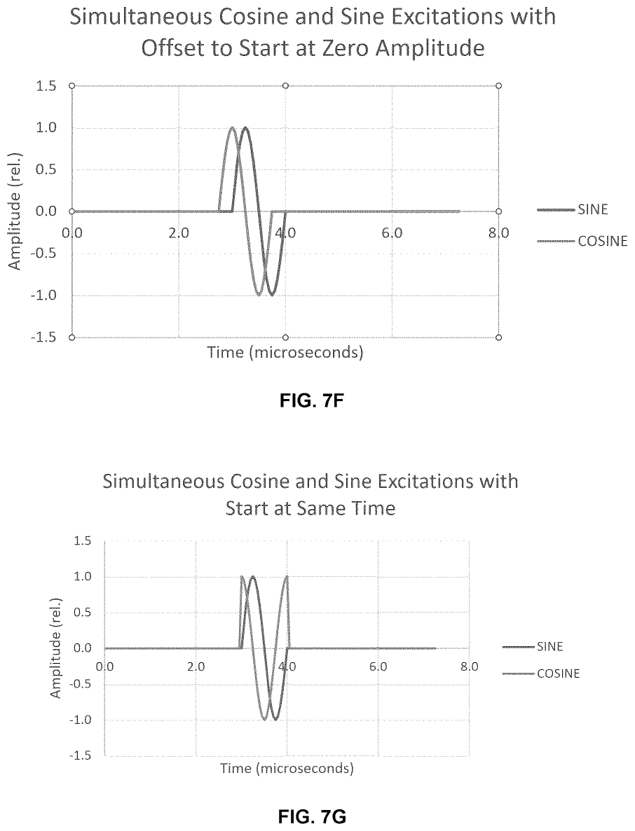

As illustrated in FIG. 7A, any achievable phase within the cluster of elements can be engineered using the combination of the two bias apertures and the quadrature delivery of a signal to the cluster. The figure schematically shows, via trigonometric identities, how the phase on any element is simply the arctan of the ratio of the bias amplitude on the element, where the ‘a’ amplitude is associated with a first signal excitation (e.g. a “cosine” excitation) of the cluster and the ‘b’ amplitude is associated with the same signal delivered in quadrature (e.g. the “sine” excitation of the cluster).

The individual excitations S_{cosine} and Sₛᵢₙₑ, delivered in quadrature to a given element (m,n) in the cluster with the respective application of the two bias apertures a_{m,n} and b_{m,n}, in the example case of unity signal amplitude, is therefore given by:

$${S}_{cosine} ( t ) = {a}_{m , n} cos ( 2 π {f}_{op} t )$$

$${S}_{sine} ( t ) = {b}_{m , n} sin ( 2 π {f}_{op} t ) ,$$

where fₒₚ is the driving signal frequency, resulting in a net synthetic excitation having the following amplitude and phase:

$${A}_{m , n} = sign ( {a}_{m , n} ) \sqrt{{a}_{m , n}^{2} + {b}_{m , n}^{2}}$$

$${φ}_{m , n} = {tan}^{- 1} ( - \frac{{b}_{m , n}}{{a}_{m , n}} ) .$$

The net phases that arise from simultaneous quadrature signal excitation of a sub-element divided in the signal dimension, with separate bias voltages applied to each sub-element based on the example case of three bias values, expressed as a_{m,n}={+1,−1,0} and b_{m,n}={+1,−1,0}, are illustrated in the table shown in FIG. 7B. As the table shows, the net amplitude from the element is expressed as the square root of the sum of the squares of the amplitudes on the high voltage lines.

As can be seen in the figure, when only using three distinct bias values of 0, +V and −V, a total of eight different effective phases (resulting from the net synthetic excitation of the elements) are available and nine possible states (including the null state) are achievable given orthogonality of the signal excitations and separate bias controls. This is because the high voltage amplitude on the sine excitation is completely independent from the cosine excitation, which enables the net synthetic phase from each element to vary by more than just 0 degrees and 180 degrees.

When considering the implementation of this scheme across an entire 2D array, this approach appears to offer a significant benefit when compared to a conventional row-column array implementation capable of generating only two phases. However, the additional amplitude emanating from elements that have both the sine and cosine sub-elements biased can lead to additional energy off-axis. Indeed, as shown in FIG. 7B, in some cases, there is a square root of 2 increase in net amplitude from the element when both bias lines are applied.

This issue may be circumvented by utilizing, for example, the electrostrictive characteristics of the material or the CMUT characteristics on the membrane (e.g. bias voltage). The polarization strength in an electrostrictor is related to the bias amplitude. Eventually, the polarization strength saturates with a high enough DC bias voltage; however, at lower bias voltages the polarization strength is reduced such that the element may be shaded or apodized without affecting the element phase. Similarly, CMUTs are bias sensitive devices. The DC bias is used to provide a restoring force on the capacitive membrane, balancing the electrostatic force created when exciting the membrane with AC voltage. The DC bias can be used to control the electromechanical efficiency of the CMUT (i.e. sensitivity can be controlled with DC bias level). When the DC bias is applied, the membrane is pulled toward the bottom substrate. If the electrostatic force pulling the membrane down overcomes the restoring force of the membrane, the membrane will collapse onto the bottom substrate. This threshold voltage is called the collapse voltage. For maximum efficiency, a CMUT cell should be operated near the collapse voltage. A negative bias voltage also acts by pulling the membrane toward the bottom substrate. In either the negative bias or positive bias cases the AC excitation voltage surfs on top of the DC bias and the combination determines the polarity of the pulse produced. If a positive DC bias is applied, the combination of the bias and positive portion of the AC voltage produces a positive membrane deflection. If a negative DC bias is applied, the combination of the negative bias and the positive portion of the AC voltage will start as a net negative and create a negative deflection and a pulse with negative polarity.

For example, it is conceivable to employ a bias line with three of more different amplitude levels (e.g. resulting in a total of at least five different distinct bias levels), such as the following example bias levels that yield additional choices based on the DC voltage polarity:

$${a}_{m , n} = \{ + 1 , + 0.707 , - 0.707 , - 1 , 0 \}$$

$${b}_{m , n} = \{ + 1 , + 0.707 , - 0.707 , - 1 , 0 \}$$

The additional bias levels allow the net amplitude associated with the quadrature excitation of both sub-elements to be constant across the aperture for different bias aperture implementations, as shown in FIG. 7C.

A desired phase delay in the bias dimension may be calculated using the distance formula without considering the element position in the azimuth dimension. The time delay for the elements in bias dimension is calculated using the distance formula:

$${t}_{focus} = \sqrt{\frac{{( {x}_{focus} - {x}_{element} )}^{2} {( {y}_{focus} - {y}_{element} )}^{2} + {z}_{focus}^{2}}{{v}_{tissue}}}$$

where t_{focus} is the time to the focus, vₜᵢₛₛᵤₑ is the velocity of sound in tissue, x_{focus} and y_{focus} are focus positions, xₑₗₑₘₑₙₜ and yₑₗₑₘₑₙₜ are positions of the element in the array, and z_{focus} is focus position in depth. The t_{focus} is related to the phase through the operational frequency. This relationship for a Fresnel aperture with 0 degrees and 180 degrees can be expressed as:

$${S}_{bias} = sign [ mod ( φ + offset , - 2 π ) - π ]$$

In the present example embodiment involving the use of a set of discrete bias values that are applied to the sub-elements, the phase calculated from the distance formula may be compared to the possible discrete phases permitted with the multiple bias levels. For example, if a chosen implementation allows for five different bias levels as in FIG. 7C, then bias level assigned to a given sub-element may be determined using the lookup table shown in FIG. 7D. For example, for a desired phase of 87 degrees, FIG. 7D indicates that because 87 degrees falls between 67.5 degrees and 112.5 degrees, the amplitudes assigned to the sine and cosine sub-elements are +1 and 0 respectively.

It will be understood that the use of shading or apodization on the bias dimension may be extended beyond just three amplitudes (five bias levels) shown in FIGS. 7C and 7D. For example, an implementation may be configured to employ four different bias amplitudes (seven different bias levels) with the following available options:

$${a}_{m , n} = \{ + 1 , + 0.866 , + 0.5 , - 0.5 , - 0.866 , - 1 , 0 \}$$

$${b}_{m , n} = \{ + 1 , + 0.866 , + 0.5 , - 0.5 , - 0.866 , - 1 , 0 \}$$

This example configuration increases the number of distinct phase angles from eight to twelve, as shown in FIG. 7E. According to such implementations, the number of distinct fine phase values is only limited by the number of possible bias levels.

It will be understood that the quadrature signals may be generated by multiple methods, provided that the excitations are orthogonal. This can be achieved, for example, by delaying a cosine excitation relative to an excitation by pi/2, as shown in FIG. 7F, or, for example, by exciting at the same time with sine and cosine waveforms, as shown in FIG. 7G.

On receive, the delay is added again. This is similar to the approach on the four transmit-receive sequence with the exception that the pi/2 delay does not have at the same time.

When employing the present example in which the coarse-delayed signals are only delivered to the clusters, as opposed to individual elements of the array, the amount of signal reduction is dependent on the number of elements per cluster. For simplicity, suppose the number of elements in a cluster is constant. If the 2D array is N by M where N is the number of rows and M is the number of columns, then the number of dedicated system channels ‘S’ is calculated as:

$$S = ( N * M ) / ( u * v ) ,$$

where u is the number of rows in the cluster and v is the number of columns in a cluster. The larger the cluster size, the greater the susceptibility to phase wrapping and thus a greater number of synthetic sub-apertures within each cluster will be necessary to minimize the error. This will be a trade-off between minimizing system channel count and required volume/frame rates.

If this technique is extended to a wide bandwidth excitation, then either an abbreviated/windowed cosine or sine excitation is at the element where the excitation signal is delayed relative to the excitation signals at the other clusters to focus at an intended point.

It is noted that the focus may be virtual to enable plane wave imaging. The remaining time delays for each individual elements are assigned as a phase using the bias lines to minimize the time delay error at each element.

Although it was noted above that the effective time delay at the cluster may be determined by the mean of required delays on the elements in the cluster or other means, this is not required. For example, it may be advantageous to keep some elements ‘off’ by either not biasing the elements or disconnecting them within the cluster due to phase wrapping and perform an additional synthetic aperture technique.

For example, in such a case, calculating and sorting the time delays for each element within the cluster (subarray) allows multiple, independent transmits where one portion (subaperture) of the cluster is ‘on’ with the appropriate bias and the remainder of the cluster is ‘off’. The portion of the cluster that is “off” may be unbiased or disconnected (i.e. an open circuit which prevents any current from flowing, this can be thought of as free or clamped in the thickness dimension, which may be advantageous for crosstalk performance). The elements that are ‘on’ have their time determined by the mean of the time of the elements that are ‘on’. Next, the appropriate bias level is assigned to make up for any remaining time delay. This then continues for the subset of cluster elements that were ‘off’, by turning those elements ‘on’, turning the 1st elements off, and using another mean delay. This method, or variations thereof, can be employed to improve or perfect beamforming at the intended focus.

The need for additional synthetic apertures can increase with the number of elements in a cluster and the steering angle. For example, a 256 by 256, 15 MHz array with half wavelength element pitch in both the azimuth and elevation dimension may be defined with a cluster size of 8 by 8. Therefore, the array has 65,536 individual elements, but because of clustering only 1,024 transmit/receive channels are required. A case is considered in which the focus is at x=0 mm, y=17.3 mm, and z=30.0 (steering of 30 degrees). The cluster in the lower left corner of the array has a center of x=−6.2 mm and y=−6.2 mm. Using the distance formula, the distance to the focus is 38.6 mm. If the mean distance from the entire cluster is removed from the position of individual elements, then the remaining distances can be calculated.

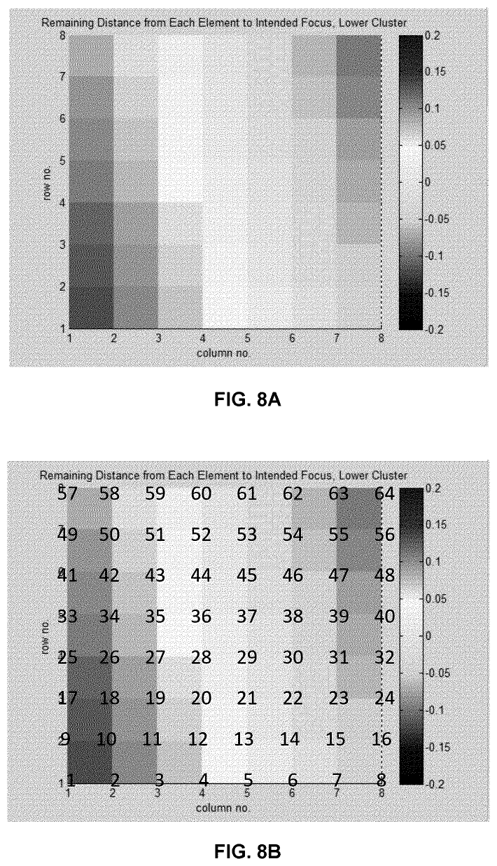

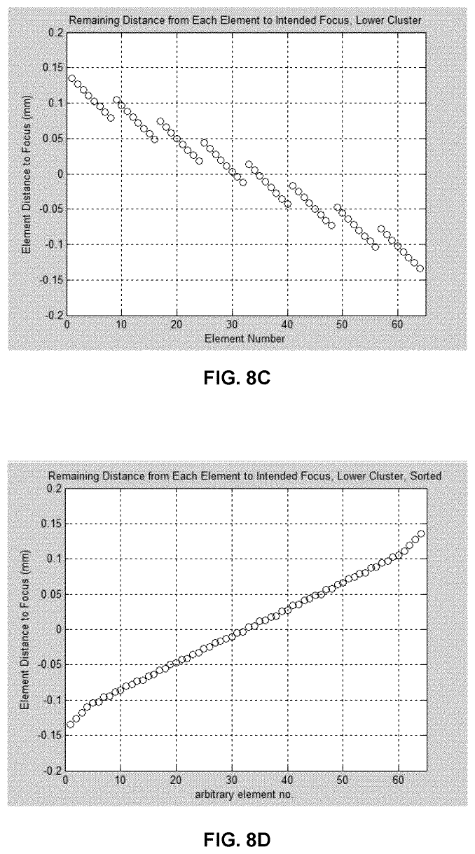

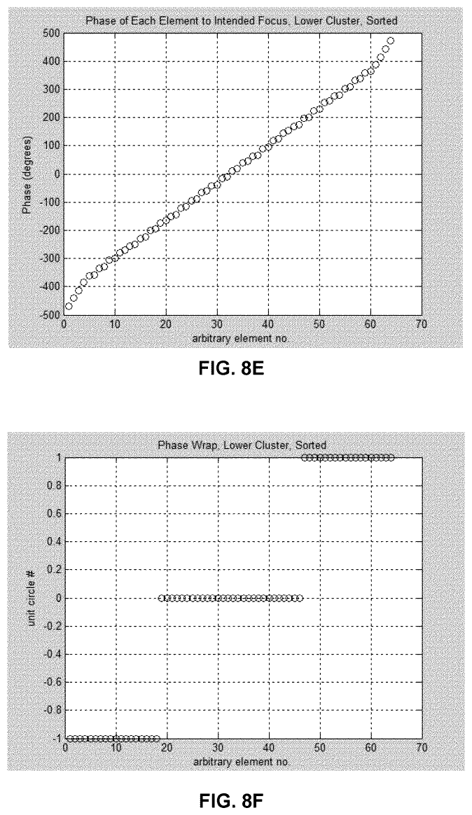

FIG. 8A shows the remaining distance from the 64 elements within the cluster in millimeters. FIG. 8B shows the labels for the elements within the cluster numbered from 1 to 64. If those distances are plotted, then the result is shown in FIG. 8C. FIG. 8D shows the distances sorted from high to low. The phase is determined by the wavelength of the distance remaining which is shown in FIG. 8E.

In this case, the difference between the maximum phase and minimum phase is approximately 940 degrees which is equivalent to 2.6 revolutions around the unit circle. Therefore, if only one delay was used with the corresponding bias for sine and cosine, the wavefronts from some elements would arrive early or late at the focus.

FIG. 8F shows that some of the elements when the phase is applied will still arrive one wavelength early and some elements will arrive one wavelength late. For example, 18 elements the wavefront arrives early, 18 elements the wavefront arrives late, and 36 elements the wavefront arrives on time. To eliminate this error, two additional quadrature apertures may be set up where the system channel delay is shifted in time by plus and minus one wavelength. When these elements are in use, then the other elements are ‘off’. Essentially, the phase wraps determine the number of additional quadrature transmit-receive events that need to occur.

It is important to note that the number of phase wraps will vary depending on the amount of steering. Therefore, the number of additional synthetic apertures required may vary depending on the focal location. Furthermore, it is possible to design the synthetic apertures to obtain a balance between the number of elements participating in transmit and receive. For example, in the previous example, one set used 18 elements within the cluster, another set also used 18 elements, and the set from the original cluster delay used 36 elements. If, to minimize phase error, three synthetic aperture sets are used for an 8 by 8 cluster, then the system delays could be set such that there would be two sets of 21 elements and one set of 22 elements used for each synthetic aperture. This configuration minimizes the variation in SNR between the synthetic apertures within the cluster.

As an additional example of phase error across the cluster, a quad cluster array is designed with the following attributes:
Operating frequency: 5 MHz
Pitch: Half wavelength (0.15 mm)
Size: 96 rows and 96 columns
Cluster size: 16 elements, 4 by 4
Total Element Count: 9,216
Aperture Size: 14.4 mm by 14.4 mm
Required System Channels: 576
System Uses Average Delay Across Cluster

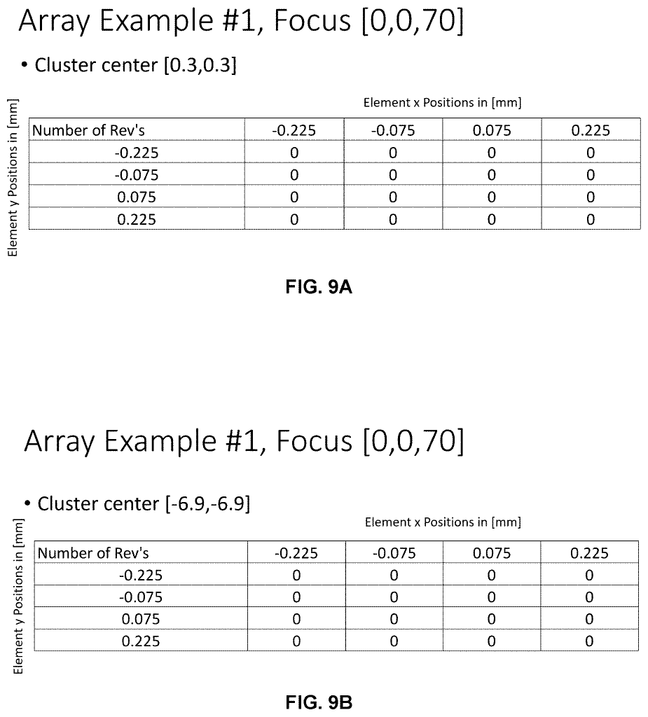

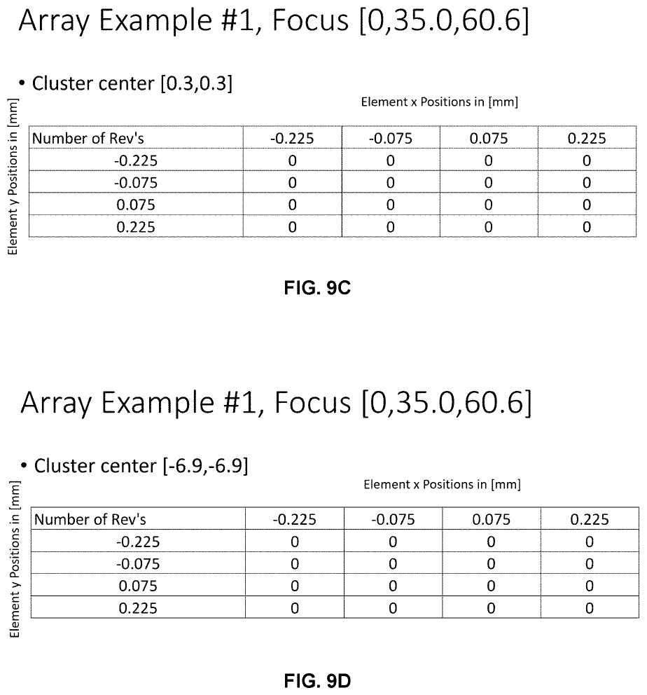

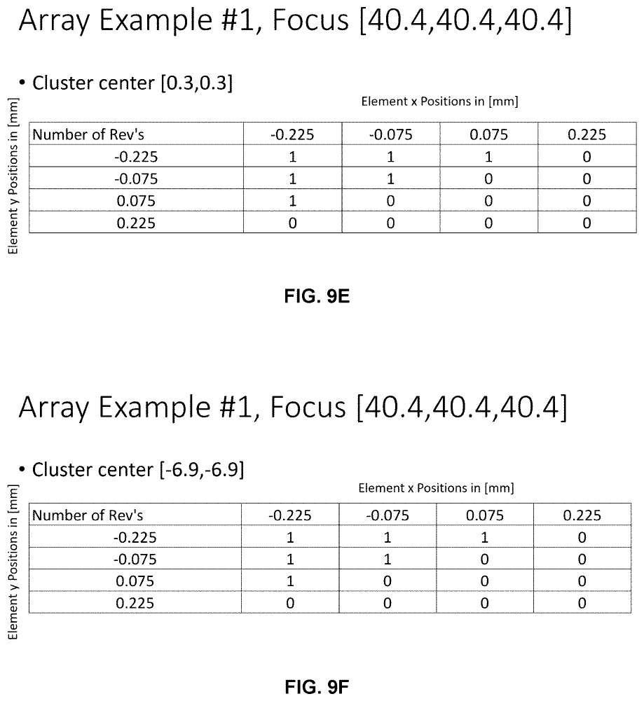

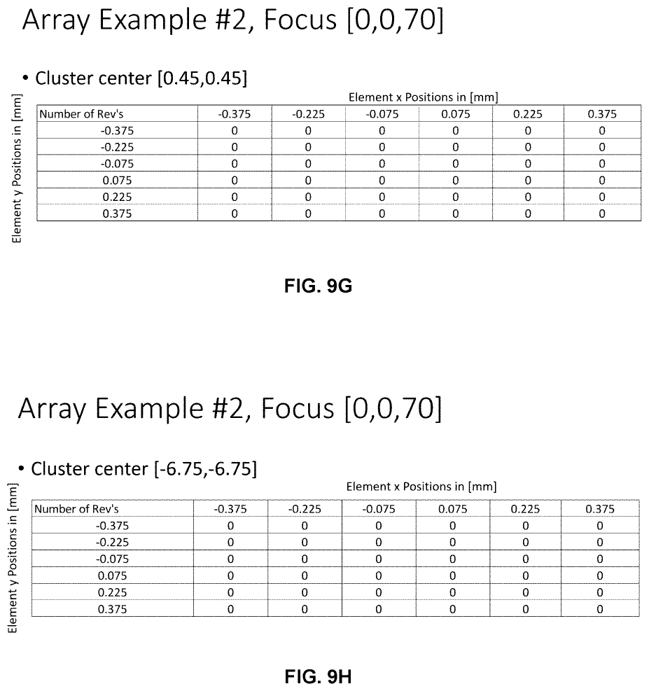

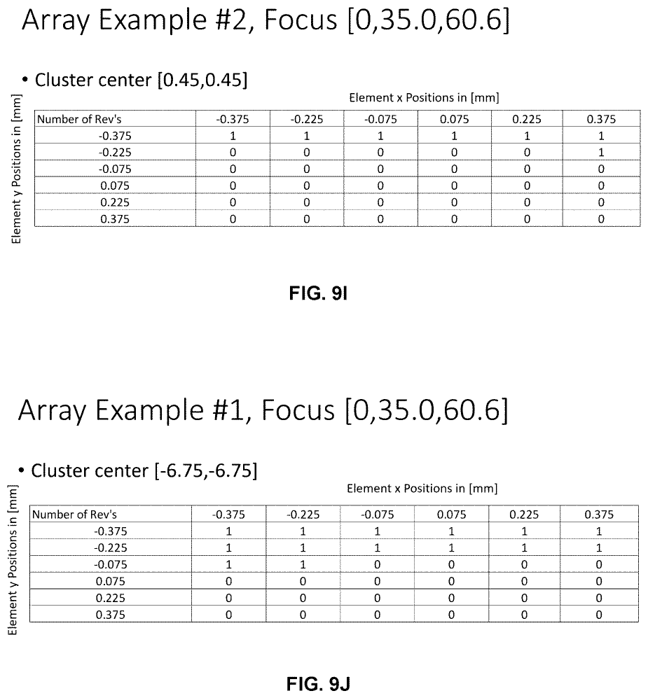

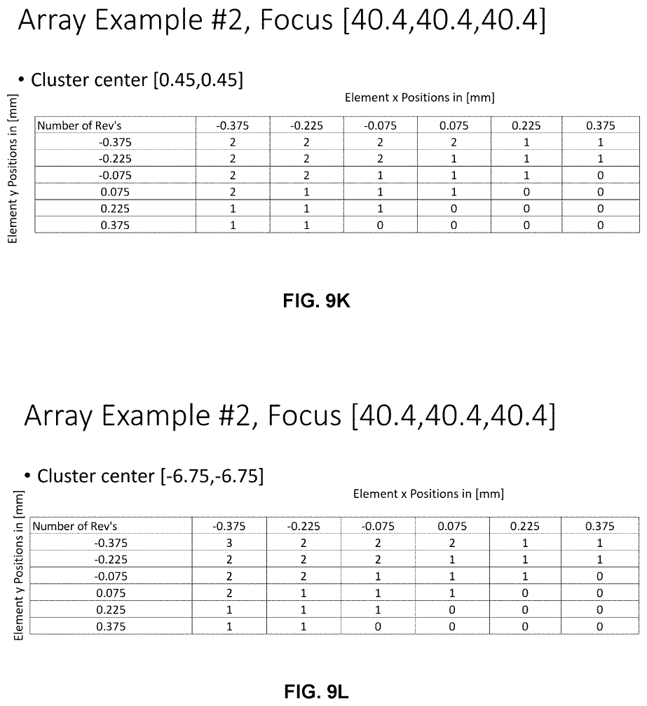

The focus initially resides at x=0 mm, y=0 mm, z=70 mm. FIGS. 9A and 9B show the number of wraps around the unit circle for a cluster near the center of the array x=0.3 mm, y=0.3 mm and one at the corner x=−6.9 mm, y=−6.9 mm. In the case of a straight ahead focus, there is no phase wrapping.

If the beam is steered 30 degrees to x=0 mm, y=35.0 mm, z=60.6 mm where the total focal length remains 70 mm, the center cluster (FIG. 9C) as well as the corner of the array (FIG. 9D) show no phase wrap. If the beam is steered 45 degrees in theta and phi, both the cluster near the center of the array as well as the one near the opposite edge of the focus show at least one phase wrap (FIGS. 9E and 9F).

In another example of phase error across a cluster, the same array is considered, but with a larger cluster size:
Operating frequency: 5 MHz
Pitch: Half wavelength (0.15 mm)
Size: 96 rows and 96 columns
Cluster size: 36 elements, 6 by 6
Total Element Count: 9,216
Aperture Size: 14.4 mm by 14.4 mm
Required System Channels: 256
System Uses Average Delay Across Cluster

The focus initially resides at x=0 mm, y=0 mm, z=70 mm. FIGS. 9G and 9H show the number of wraps around the unit circle for a cluster near the center of the array x=0.3 mm, y=0.3 mm and one at the corner x=−6.9 mm, y=−6.9 mm. In the of a straight ahead focus, there is no phase wrapping just like the 4×4 cluster.

If the beam is steered 30 degrees to x=0 mm, y=35.0 mm, z=60.6 mm where the total focal length remains 70 mm. In this case, the center cluster (FIG. 9I) as well as the corner of the array (FIG. 9J) show at least one phase wrap with the corner cluster showing that a greater number of elements were affected. If the beam is steered 45 degrees in theta and phi, both the cluster near the center of the array as well as the one near the opposite edge of the focus show at multiple phase wraps (FIGS. 9K and 9L). In this case, it may be best to utilize four sub-apertures within the array to minimize phasing errors when acquiring image vectors at large steering angles.

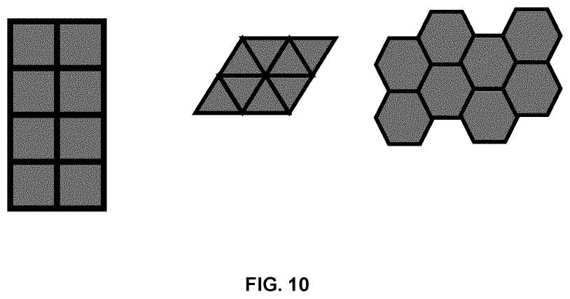

Although many of the examples herein show individual transducer elements as squares of uniform shape, it will be understood that this is not a requirement. As illustrated in FIG. 10, it will be understood that a cluster need not be limited to a square or rectangular shaped group of elements and the cluster does not need to be square.

As shown in the figure, elements may be shaped as triangles, hexagons, squares, or another shape. For example, a group of 16 elements could be arranged as 8 by 2, 2 by 8, 16 by 1, or 1 by 16 depending on the imaging application. In either case, this is a type of cluster. The clusters may be advantageously organized when steering/focusing where the directional vector includes both x and y.

Furthermore, it will be understood that the shape of the individual elements does not need to be uniform throughout the entire array. Similarly, the elements do not have to have a uniform periodicity in the azimuth or elevation direction to apply this method.

It is noted that although many of the present examples show that every cluster is the same size and consists of the same number of elements, this is not a requirement. In other words, some arrays may benefit from having larger element clusters in the middle of the array and smaller element cluster to the edge. Similarly, some arrays may benefit from smaller element clusters in the middle of the array and larger element clusters to the edge.

It is also noted that the example individual pillars or elements shown and described herein are merely shown as examples to describe the portion of the array which has a unique bias voltage that could be applied. The element or pillar could be multiple sub-pillars (e.g., sub-diced region) or membranes or even an electrode defined excitation region on a solid piece of material (kerf-less array) where the elements are defined through separation of the conductive surface.

In some example embodiments, the preceding example methods may be employed to synthetically focus ultrasound energy to a plurality of adjacent focal locations without modifying the first and second bias apertures. For example, an additional first transmit operation and an additional second transmit operation may be performed to synthetically focus ultrasound energy at a second real focus residing proximal to the first real focus. The additional first transmit operation is performed using the first transmit bias aperture and the additional second transmit operation is performed using the second transmit bias aperture, such that the second real focus is obtained by modifying the coarse transmit subarray delays applied to the transmit signals without modifying the first transmit bias aperture and the second transmit bias aperture. In some example embodiments, additional first and second transmit operations may therefore be performed to synthetically focus ultrasound energy at a plurality of focal locations within a selected sector by modifying the coarse transmit subarray delays applied to the transmit signals without modifying the first transmit bias aperture and the second transmit bias aperture.

Although the preceding example embodiments have been disclosed within the example context of the synthetic transmission, many example embodiments also employ the cluster-based array configuration to perform corresponding receive operations, for example, for imaging applications.

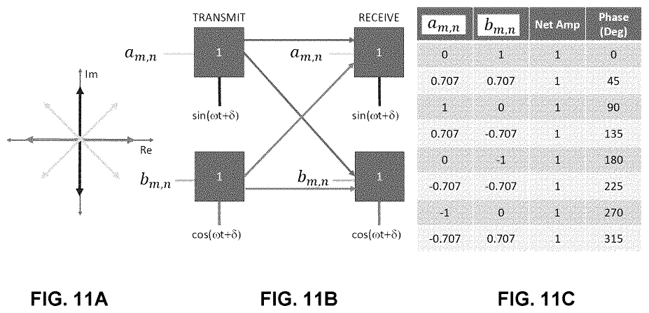

An example implementation of a synthetic transmit/receive embodiment involving four transmit/receive events is shown in FIGS. 11A to 11C, with FIG. 11C showing example combinations of bias values that result in net phases for constructing the cosine and sine bias apertures to achieve a desired net synthetic phase profile.

As shown in FIG. 11B, the example four-pulse transmit/receive sequence is implemented using the following bias aperture combinations: TX1/RX1=sine/sine, TX2/RX2=sine/cosine, TX3/RX3=cosine/sine, TX4/RX4=cosine/cosine (preferably, TX4/RX4 is performed prior to TX3/RX3 to limit aperture switching). The cosine aperture is employed, in transmit, with excitation signals that are in quadrature with those that are applied in transmit with the sine aperture. When the cosine aperture is employed in receive, the resulting receive signals are delayed by a time delay corresponding to a phase delay of π/2. The four transmit/receive events may be applied in any order.

The use of both the cos and sine apertures in both transmit and receive generates an effective (synthetic) bias aperture with multiple phases levels, as in the preceding example embodiments. For each of the sine and the cos apertures, the bias amplitude can be calculated in the same manner as previously described.

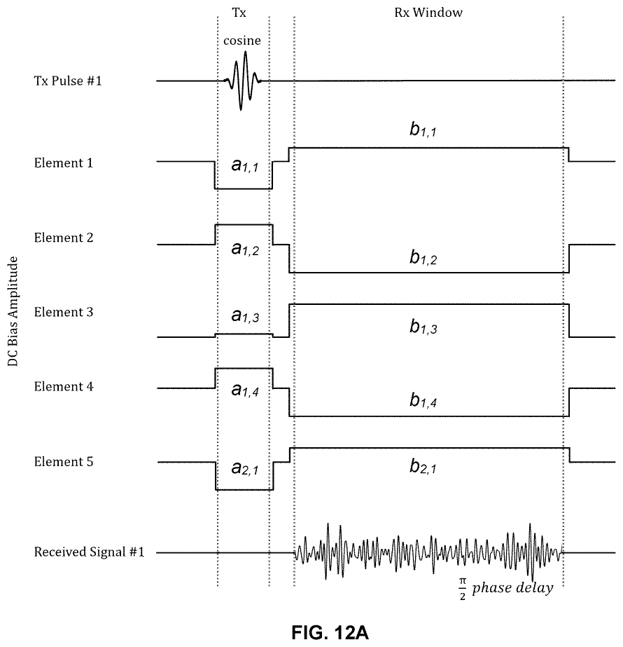

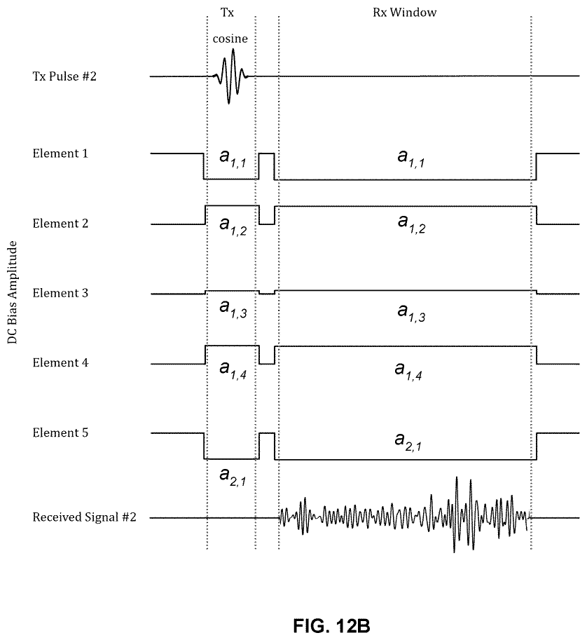

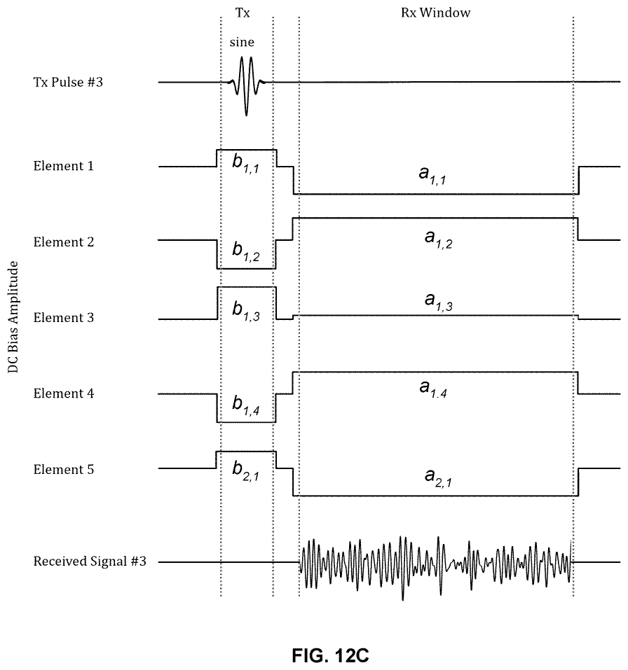

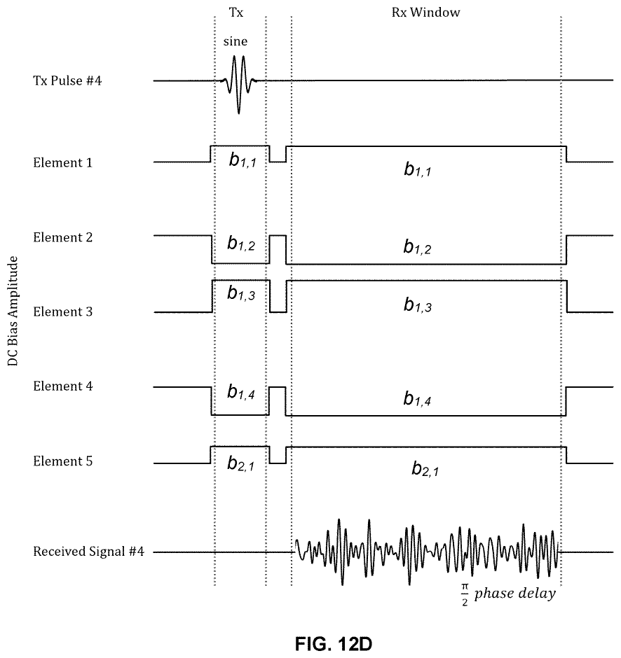

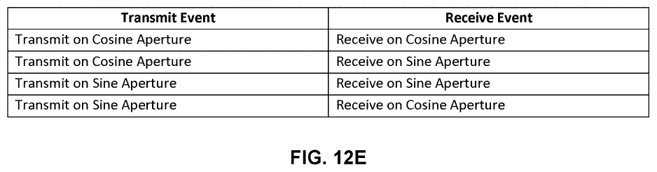

FIGS. 12A-12D illustrate the transmit pulse sequence for an example embodiment showing the first 5 bias electrodes in an example cluster. Referring first to FIG. 12A, a cosine/sine transmit event is illustrated. The first row of the figure shows the timing of the transmit pulse delivered to the signal electrode of the cluster. This transmit signal set is referred to as a “cosine” transmit signal and is delivered while applying the cosine bias aperture to the bias electrodes, as shown in the figure by the a_{1,1} to a_{2,1} bias levels applied to the bias electrodes. The received signal is shown at the bottom of the figure and is referred to as a “sine” signal and is received while applying the sine bias aperture to the bias electrodes, as shown in the figure by the b_{1,1} to b_{2,1} bias levels applied to the bias electrodes. The received sine signal is delayed by a time delay corresponding to a phase shift of π/2, so that the sine signal is detected in quadrature with the cosine signals that are received without delay, as described in further detail below.

FIG. 12B shows a cosine/cosine transmit event, where the first row of the figure shows the timing of the cosine transmit pulse delivered to the signal electrode while applying the cosine bias aperture to the bias electrodes, as shown in the figure by the a_{1,1} to a_{2,1} bias levels applied to the bias electrodes. The received signal is shown at the bottom of the figure and is referred to as a “cosine” signal and is received while applying the cosine bias aperture to the bias electrodes, as shown in the figure by the a_{1,1} to a_{2,1} bias levels applied to the bias electrodes. Unlike the received sine signal in FIG. 12A, the received cosine signal is not delayed, so that it is quadrature with the received sine signals detected in other transmit/receive events.

FIG. 12C shows a sine/cosine transmit event, where the first row of the figure shows the timing of the sine transmit pulse delivered to the signal electrode while applying the sine bias aperture to the bias electrodes, as shown in the figure by the b_{1,1} to b_{2,1} bias levels applied to the elevation electrodes. As can be seen by comparing FIG. 12C to FIG. 12B, the sine transmit pulse is generated in quadrature with respect to the cosine transmit pulse. The received cosine signal is shown at the bottom of the figure and is received while applying the cosine bias aperture to the bias electrodes, as shown in the figure by the a_{1,1} to a_{2,1} bias levels applied to the bias electrodes.

FIG. 12D shows a sine/sine transmit event, where the first row of the figure shows the timing of the sine signal pulse delivered to the signal electrode while applying the sine bias aperture to the bias electrodes, as shown in the figure by the b_{1,1} to b_{2,1} bias levels applied to the bias electrodes. The sine transmit pulse is generated in quadrature with respect to the cosine transmit pulse of the other transmit/receive events. The received sine signal is shown at the bottom of the figure and is received while applying the sine bias aperture to the bias electrodes, as shown in the figure by the b_{1,1} to b_{2,1} bias levels applied to the bias electrodes.

The four sets of received signals that result from the four pulse-echo events are summed (synthetically compounded), with the application of the π/2 phase shift when the sine aperture is employed in receive, to achieve receive signals that correspond to a focus associated with the net desired receive phase profile.

While the four transmit/receive events may be performed in any order, it may be beneficial to employ a sequence order shown that minimizes the number of switches between the cosine and sine apertures, since significant switching may cause heat generation as the elements are biased to a different voltage. A non-limiting example of such a sequence is shown in FIG. 12E.

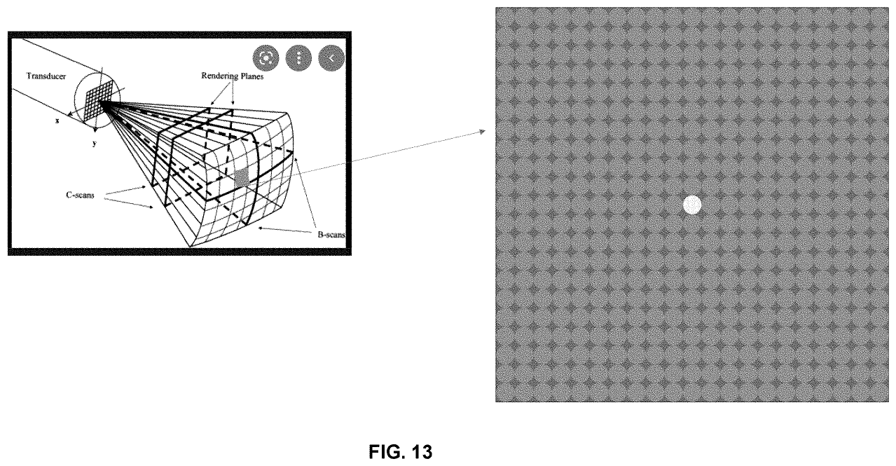

Referring now FIG. 13, a 2D array is shown, along with a potential 3D volume FOV based on multiple transmit-receive vectors. As with an 2D array or volume imaging, an important performance attribute is the achievable volume rate. The FOV shown in FIG. 13 may be separated into smaller transmit-receive sections. One of these sections is shown in orange in the FOV. These separate sections may be determined by angular size, for example 5 degrees by 5 degrees, in theta and phi if in polar coordinates or actual distances in x and y if in a Cartesian coordinate system.

In some example implementations, this region may be insonified using plane wave ultrasound. The plane waves within this region could come at different angles. After insonifying this region at different angles, the present example quadrature technique may be applied simultaneously to calculate the pulse-echo response at a multitude of points within the region. In this scenario, the bias aperture is changed between transmit events to switch between the cosine and sine apertures or to just slightly move the focus for better compounding results.

In the case of quad cluster, a cosine excitation and a sine excitation is necessary for every angle and for the cosine and sine receive apertures for a total of four transmits. Initial time delays and appropriate biases are applied to each cluster for the sine and cosine transmit apertures to generate a wavefront at the intended angle. If eight different plane wave angles are used, then a total of 32 transmits are required to account for the sine and cosine transmit and receive apertures in the case of synthetic quad cluster.

A synthetic focus may be generated at any point where the plane waves overlap. A time delay is applied to the cluster signals to ensure that the plane waves pass through the point of interest at the same time. In order to create a two-way focus, time delays and appropriate biases for the sine and cosine receive apertures are also applied in receive to focus the transducer at the points interest in the region where the plane waves overlap.

To obtain volume imaging, sine and cosine plane wave transmits are used with corresponding sine and cosine receive apertures. Receive biases for the cosine and sine apertures are applied to focus the beam at a point along with appropriate time delays on the cluster lines.

FIG. 13 shows an enlarged region of the section within the pyramidal volume. If the intention is to focus, upon receive, at the yellow dot in the enlarged region, appropriate cosine and sine biases are applied within each cluster upon receive such that when the appropriate time delays are applied to the cluster, a focused beam, upon receive, can be generated at the intended location (yellow dot).

The present simulation enables a determination of how many lines could potentially be fit within one section, given just one set of bias patterns employed in receive. To make this determination, a one-way PSF was simulated by fixing the bias pattern to a straight ahead focus for the example quadrature cluster transducer described in the Example below. Next, the delays were modified to steer the dynamic receive beam focus. In one case, the beam was steered 2 degrees which implies 441 beams could fit within a region of +/−2 degrees in theta and phi if the sampling is 0.2 degrees between beams without requiring a change in the bias pattern. This number of beams, 441, is what is represented in FIG. 13. Therefore, if the FOV has a theta and phi angle of 80 degrees respectively, then a total of 441 sectors could be imaged using this method. If the amount of steering was increased to +/−4 degrees, then a total of 1681 beams could be generated within one sector.

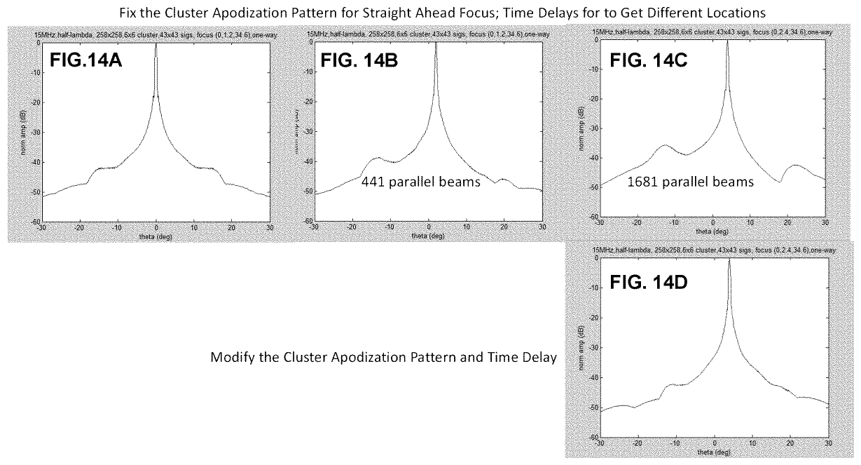

FIGS. 14A-14C shows the PSF results for these cases. Overall, the PSF appears to be reasonably ideal with some slight increase in the clutter level when attempting to steer the beam to 4 degrees.

FIG. 14A shows the resulting one-way beam response using this method. Although the bias pattern is ideally suited to focus at the central dot, it is possible to steer the beam to other locations within the enlarged region by adjusting the time delays applied on each cluster to minimize the focusing error. FIG. 14B shows the resulting one-way beam response using this method where the beam is steered 2 degrees (a dot at the periphery of the zone shown in FIG. 13). FIG. 14C shows the resulting one-way beam response using this same method where the beam is steered 4 degrees. FIG. 14D shows the result if the apodization function is allowed to change and shows that the PSF with the original bias pattern is not far off from the ideal bias pattern.

Overall, the one-way responses for steered cases shows that although the bias pattern is not ideal, adjusting the time delays to minimize the focusing error still yields sufficient focused beam performance. This allows for multiple receive beams to be generated from the bias patterns from one focus. For example, in the discussed example, 1681 receive beams could be generated by just using the bias patters for the straight ahead focus (central dot). There are additional performance advantages as previously described if the plane wave transmit is concentrated to just one region of the entire volume. The number of parallel receive beams that can be generated can be used to increase the volume rate performance of the quad cluster transducer.

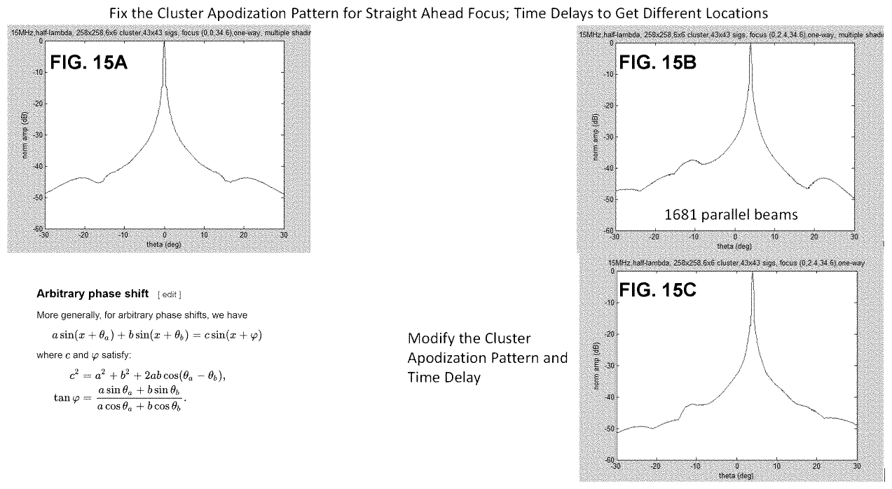

FIGS. 14A-14C have demonstrated that multiple receive beams are possible by just adjusting the time delays on each cluster without any bias modifications. However, because multiple transmit plane waves are used for the transmit focus, it is possible to introduce multiple receive bias patterns to generate a coherent compounded image. FIG. 15A shows the resulting one-way response for the straight ahead focus which combines the responses using the straight ahead focus and the slightly steered focus (4 degrees). A similar method may be used for the steered focus which is shown in FIG. 15B. FIG. 15C shows the same response for beam steered 4 degrees if only the ideal bias patterns are used. FIGS. 15A and 15B show that coherently compounding multiple bias patterns for the same focal region leads to slightly different net beam responses.

It is important to point out that the bias patterns for the different focal locations may be mixed for the sine and cosine apertures. In other words, it is not necessary to receive with the sine and cosine bias apertures for the straight ahead focus and also the sine and cosine bias apertures for the steered case. For example, in one case the bias pattern for the straight ahead sine aperture could be mixed with the steered case cosine aperture to obtain some compounding benefit. Also, the compound does not have to happen within the same plane, the focus could also moved in depth to receive some compounding benefits.

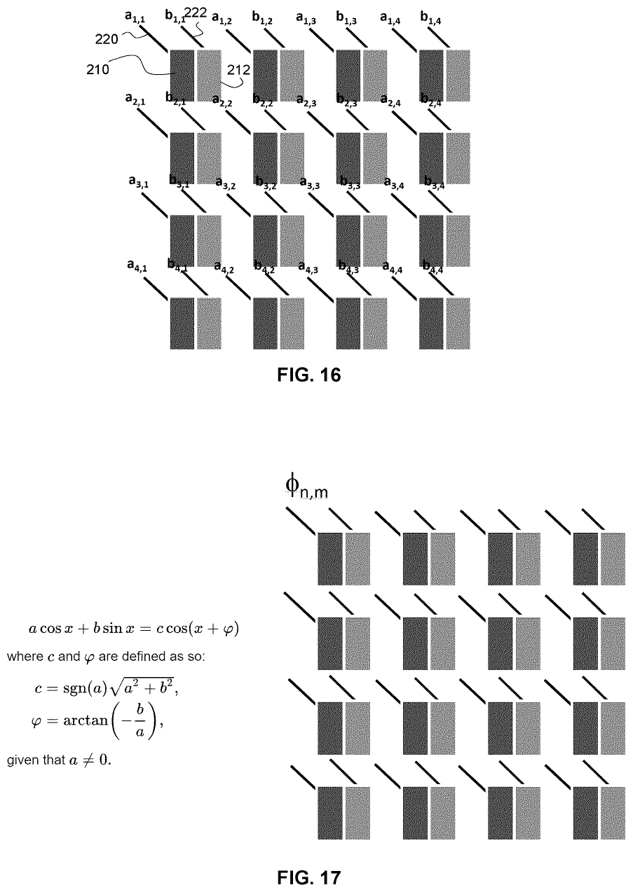

While the preceding example embodiments pertain to synthetic transmit operations using quadrature excitation with signals delayed by per-cluster coarse time delays and per-cluster bias apertures for fine phase control, FIG. 16 illustrates another example embodiment in which the first and second quadrature transmit operations may be performed simultaneously for generating a real transmit focus, as opposed to a synthetic transmit focus. Such an example embodiment may be beneficial for applications involving focused ultrasound, such as, for example, ultrasound therapy, and may also be employed for imaging applications.

Referring now to FIG. 16, an example ultrasound array is illustrated in which each ultrasound element includes a first sub-element 210 and a second sub-element 212 residing adjacent to one another, each sub-element being capable of acoustic transduction under application of a bias. A first set of bias conductive paths is provided, each first bias conductive path 220 being in electrical communication with a respective bias electrode of a first sub-element, along with a second set of bias conductive paths, each second bias conductive path 222 being in electrical communication with a respective bias electrode of a second sub-element, thereby enabling each first and second sub-element to be individually biased. First and second sets of signal conductive paths are also provided (not shown in FIG. 16, as they reside on the opposite side of the array). Each first signal conductive path delivers a respective signal to a respective set of first sub-elements of a cluster (subarray) of ultrasound elements, and each second signal conductive path delivers a respective signal to a respective set of second sub-elements of a cluster (subarray) of ultrasound elements, thereby enabling the respective signals to be applied, in quadrature, to each first sub-element and second sub-element of the respective cluster of ultrasound elements.

The first and second transmit operations are preformed simultaneously. The first transmit operation is performed by delivering a first set of time-delayed transmit signals to the first set of signal conductive paths while applying a first transmit bias aperture to the first set of bias conductive paths, with each transmit signal of the first set of time-delayed transmit signals being delivered to a respective cluster with a respective per-cluster coarse transmit delay associated with the transmit phase aperture. Likewise, the second transmit operation is performed by delivering a second set of time-delayed transmit signals to the second set of signal conductive paths while applying a second transmit bias aperture to the second bias conductive paths, with the second set of time-delayed transmit signals being generated in quadrature relative to the first set of time-delayed transmit signals.

The first transmit bias aperture and the second transmit bias aperture are configured (as described above) such that when the first transmit operation and the second transmit operation are simultaneously performed, each pair of first and second sub-elements generates a net fine phase delay associated with the transmit phase aperture (as schematically illustrated in FIG. 17), such that a combination of the per-cluster coarse transmit subarray delays and the per-sub-element fine phase delays generate or approximate the transmit phase aperture.

It will be understood that the example embodiment illustrated in FIGS. 16 and 17 may be adapted for imaging applications by performing receive operations according to any of the preceding example embodiments, with the difference being that the four synthetic transmit and receive operations may be condensed into two synthetic transmit and receive operations, each transmit and receive operation involving the simultaneous quadrature transmission and detection using both sets of sub-elements.

For example, the two transmit operations can each be performed according to the transmit operation described above, while the first and second receive operations can differ by switching the bias apertures (and quadrature delay) among the first and second sets of sub-elements. For example, if a given transmit/receive operation is performed as (transmit using first set of sub-elements/transmit using second set of sub-elements)/(receive using first set of sub-elements/receive using second set of sub-elements), and if the transmit and receive bias apertures are TX1A, TX1B, RX1A and RX1B, then the two transmit-receive events may be performed as (TX1A/TX1B)/(RX1A/RX1B)=(sine/cosine)/(sine/cosine) and (TX1A/TX1B)/(RX1B/RX1A)=(sine/cosine)/(cosine/sine), where the underlined transmit apertures denote the application of the aperture in quadrature, and where the underlined receive apertures denote the application with the aperture with delay of the received signals by a delay corresponding to a phase delay of π/2.

In some example embodiments, the present example embodiments may be adapted to perform tissue harmonic imaging as well as filtered harmonic imaging.

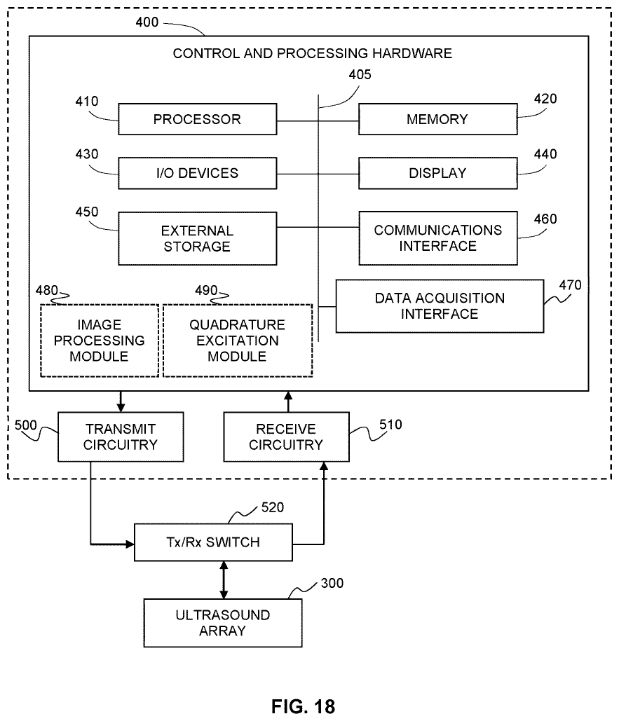

Referring now to FIG. 18, an example imaging system is illustrated for performing quadrature cluster-based excitation with an ultrasound array. The example system includes an ultrasound array 300 that includes a set of ultrasound transducer array elements (e.g. piezoelectric elements, which may be a component of an ultrasound imaging device, such as an ultrasound imaging endoscope), transmit circuitry 500 for delivering transmit voltage pulses to the ultrasound array 300, a transmitter-receiver switch 520, receive circuitry 510 for detecting receive signals from the ultrasound array 300, and control and processing hardware 200 (e.g. a controller, computer, or other computing system). The transmitter-receiver switch 520 and receive circuitry 510 are employed for imaging implementations but may be absent in transmit-only implementations, for example, in some therapeutic applications.

Control and processing hardware 200 is employed to control transmit circuitry 300 and Tx/Rx switch 520, and for processing the receive signals obtained from receive circuitry 510. As shown in FIG. 18, in one embodiment, control and processing hardware 300 may include a processor 410, a memory 420, a system bus 405, one or more input/output devices 430, and a plurality of optional additional devices such as communications interface 460, display 440, external storage 450, and data acquisition interface 470.

The present example methods of performing quadrature transmission and receive via a cluster-based transducer array can be implemented via processor 410 and/or memory 420. As shown in FIG. 18, the control of the delivery of quadrature excitation transmit signals, the application of suitable bias apertures in transmit and receive, and beamforming of receive signals may be implemented by control and processing hardware 400, via executable instructions represented as quadrature excitation module 490. The control and processing hardware 400 may include and execute scan conversion software (e.g., real-time scan conversion software) or other image processing functionality as represented by image processing module 480.

The functionalities described herein can be partially implemented via hardware logic in processor 410 and partially using the instructions stored in memory 420. Some embodiments may be implemented using processor 410 without additional instructions stored in memory 420. Some embodiments are implemented using the instructions stored in memory 420 for execution by one or more general purpose microprocessors. In some example embodiments, customized processors, such as application specific integrated circuits (ASIC) or field programmable gate array (FPGA), may be employed. Thus, the disclosure is not limited to a specific configuration of hardware and/or software.

Referring again to FIG. 18, it is to be understood that the example system shown in the figure is not intended to be limited to the components that may be employed in a given implementation. For example, the system may include one or more additional processors. Furthermore, one or more components of control and processing hardware 400 may be provided as an external component that is interfaced to a processing device. For example, as shown in the figure, any one or more of transmit circuitry 500, receive circuitry 510, and Tx/Rx switch 520 may be included as a component of control and processing hardware 400 (as shown within the dashed line), or may be provided as one or more external devices.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed herein can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

The example embodiments described above may provide several benefits and/or advantages in a variety of applications. For example, complex and expensive ASICs, with specialized transmit and receive circuitry, may be reduced or eliminated. The reduction in signal channels and ability to avoid specialized circuitry may result in a reduction in heating.

The present example embodiments may also facilitate real-time volume imaging in areas with limited accessibility, such as, but not limited to, extracorporeal cardiac ultrasound imaging (UI), endocavity UI, endovaginal UI, endoscopic UI, intraoperative UI, intra-Cardiac Echo (ICE) UI, and transesophageal UI.

As the embodiments described herein do not require orthogonality to obtain the entire volume, any unique 2D plane is available to the operator with the same frame rate, unlike PZT row-column, conventional row-column, quad row-column implementations. Moreover, the present example embodiments can achieve higher SNR than row-column implementations because to perform plane wave ultrafast imaging, one can concentrate the plane wave throughout the intended focal region. This also offers substantially better SNR than the commercial matrix arrays from Philips. Moreover, it is noted that partial beamforming is done at the element level in both transmit and receive, and that standard BGA techniques may be used to enable electrical connections to bias the elements.

### EXAMPLES

The following examples are presented to enable those skilled in the art to understand and to practice embodiments of the present disclosure. They should not be considered as a limitation on the scope of the disclosure, but merely as being illustrative and representative thereof.

### Example 1: Simulation Results

Field II simulations using polar coordinates were executed for a fully sampled 2D array and a quad cluster array. The characteristics of the gold standard array are shown below:
Operating frequency: 15 MHz
Bandwidth: 80%
Pitch: Half wavelength (0.05 mm)
Size: 258×258
Total Element Count: 66,564
Aperture Size: 12.9 mm by 12.9 mm
Required System Channels: 66,564

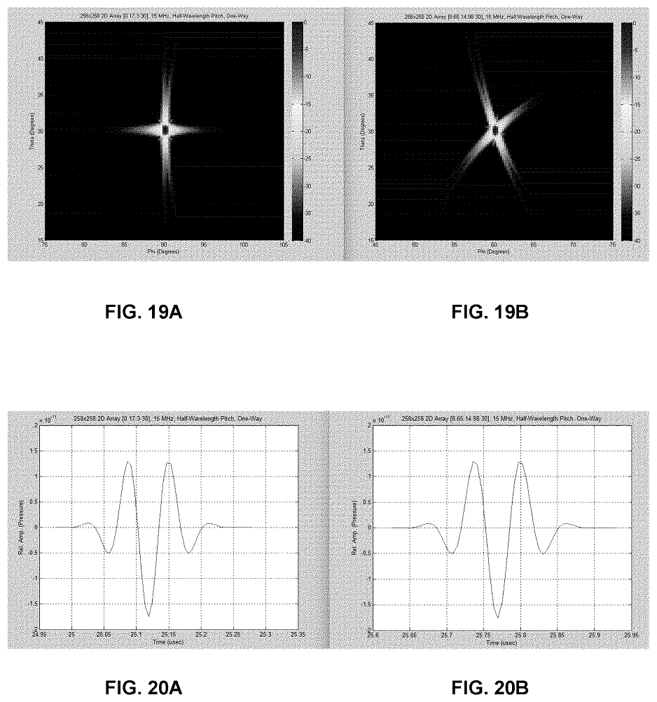

FIGS. 19A and 19B shows the resulting one-way PSF of the 2D array for two different focal conditions: Focus1, x=0 mm, 17.3 mm, 30.0 mm (theta=30 degrees, phi=0 degrees); FIG. 19A, Focus2, x=8.65 mm, 14.98 mm, 30 mm (theta=30 degrees, phi=30 degrees); FIG. 19B.

Overall, the results are as expected where most of the off-axis energy are localized within two dimensions.

FIGS. 20A and 20B show the pulse at the intended focus for the two simulations. Overall, the peak-to-peak amplitude is the same with little diminished amplitude for Focus2 where the beam is steered in both theta and phi.

The performance of the fully sampled 2D array was compared to a quad cluster array. The characteristics of the quad cluster array are shown below:
Operating frequency: 15 MHz
Bandwidth: 80%
Pitch: Half wavelength (0.05 mm)
Size: 258×258
Cluster Size: 6×6
Total Element Count: 66,564
Aperture Size: 12.9 mm by 12.9 mm
Signal Count: 43×43 (1,849 channels)

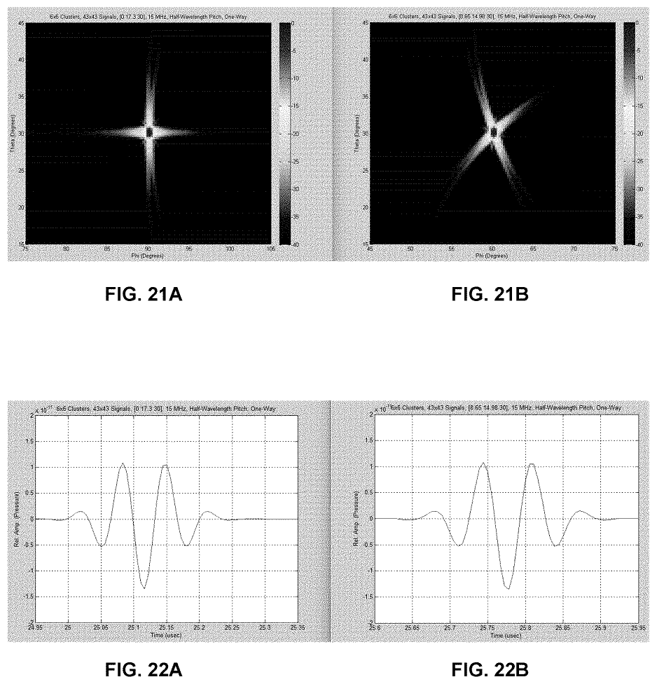

FIGS. 21A and 21B shows the one-way PSF for the same two foci captured in FIGS. 19A and 19B for the fully sampled 2D array. In this case, when comparing the surface plots, there is little or no difference between the fully sampled 2D array and the quad cluster. The only noticeable difference is between FIG. 19A and FIG. 21A where there is extra energy along the Phi dimension.

FIGS. 22A and 22B and show the transmit pulse at the focus. It is important to note that the peak-to-peak amplitude for both transmits are approximately the same (note: this is using synthetic transmit). The net result is that the quad clusters transmit is approximately 1.6 dB below the gold standard. When comparing the pulse length in FIG. 20A and FIG. 22A, overall, there is little or no visible difference.

Another simulation was performed to compare a fully sampled 2D array also at 15 MHz with an 8×8 quad cluster array. The characteristics of this 2D array were as follows:
Operating frequency: 15 MHz
Bandwidth: 80%
Pitch: Half wavelength (0.05 mm)
Size: 256×256
Total Element Count: 66,536
Aperture Size: 12.8 mm by 12.8 mm
Required System Channels: 66,536

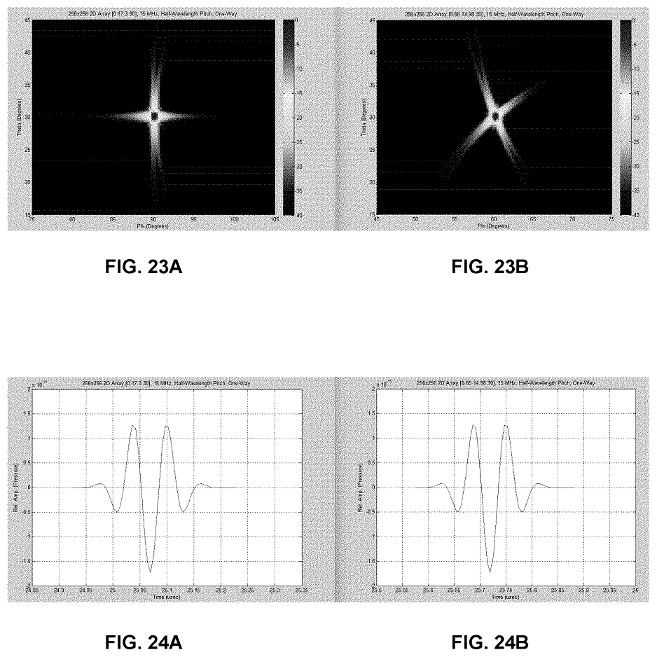

FIGS. 23A and 23B show the one-way point spread functions (PSFs) for the two different steering angles. Because the size of the aperture was only reduced by 0.1 mm in both azimuth and elevation, the PSFs are visually identical to FIGS. 19A and 19B. FIGS. 24A and 24B show the pulse at the transmit focus. Overall, the peak-to-peak amplitude is nearly the same as FIGS. 20A and 20B with the number of observable cycles equivalent to three.

These results from the fully sampled 2D array were compared to a quad cluster with characteristics shown below:
Operating frequency: 15 MHz
Bandwidth: 80%
Pitch: Half wavelength (0.05 mm)
Size: 256×256
Cluster Size: 8×8
Total Element Count: 66,536
Aperture Size: 12.8 mm by 12.8 mm
Signal Count: 32×32 (1,024 channels)

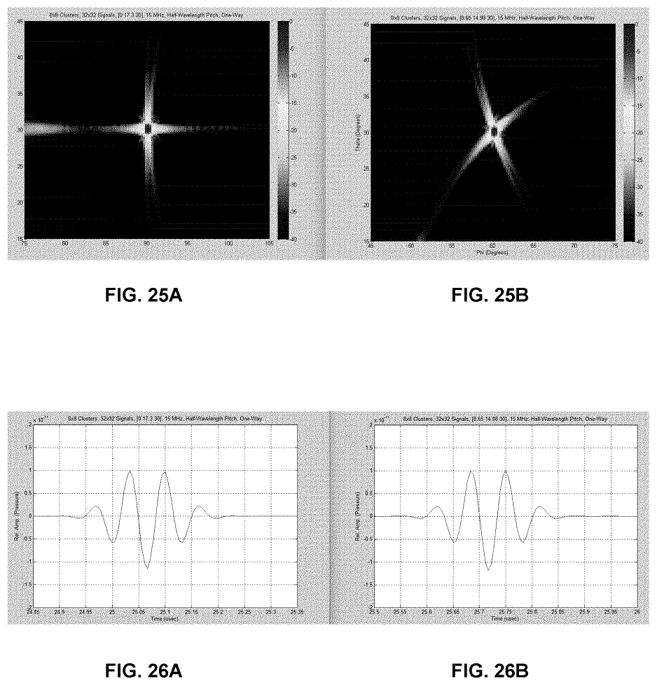

In this case, the additional energy in the phi dimension is easily observed (FIG. 25A). This extra energy is due to the phase wrapping the occurs in the larger cluster. This additional energy can be minimized using compounding as well as multiple synthetic transmits that enable an ideal phase delay to be formed. FIG. 25B also shows the additional off-focus energy which appears to be slightly lower in magnitude when compared to steering at only 30 degrees.

Regarding the pulse at the focus for this configuration, the amplitude is 3 dB below the fully sampled 2D array which may possibly be made up through transmitting at a higher power (FIGS. 26A and 26B). Also, it is noted that this is a pulse-echo response, there are added benefits to adding multiple receive waveforms at the transducer where noise is reduced by the square root of the number of receptions. In this case, because there are four receptions from four transmits, the benefit to the SNR is +6 dB which is more than sufficient to make-up the difference in transmit sensitivity. FIGS. 26A and 26B also show that the pulse length at the focus has increased when compared to the fully sampled transducer by approximately one cycle which is again attributed to the phase wrapping within each quad cluster.

### Example 2: Theory of Phase Delay Control in Synthetic Transmit/Receive Quadrature Excitation Fresnel Focusing for Tissue Harmonic Imaging

The aforementioned example embodiments are typically implemented using the same operational frequency on transmit and receive. It has been shown using conventional ultrasound diagnostic transducers that harmonic imaging improves contrast and resolution over standard imaging that transmits and receives at the same frequency.

Traditional tissue harmonic imaging (THI) may be accomplished either using a filtered technique where only one transmit is required or a pulse-inversion method where two transmits are required which are 180 degrees out of phase. Fresnel tissue harmonic imaging (FTHI) may also be accomplished with either a filtered technique or pulse inversion technique.

### Filtered Fresnel Tissue Harmonic Imaging

If using the filtered technique, four transmits are still required. However, unlike traditional THI where the filtering typically occurs on the received beamformed signal, FTHI filtering starts at the receive aperture where the Fresnel pattern is determined by the harmonic frequency in addition to filtering on the received beamformed signal.

$${v}_{r , 1} ( {x}_{1} , t , {f}_{op} ) = A \cdot cos ( 2 π {f}_{op} \cdot t + θ ) * {h}_{a , tx} ( {x}_{1} , t , {f}_{op} ) * {h}_{a , rx} ( {x}_{1} , t , 2 {f}_{op} ) * {f}_{s} ( {x}_{1} ) \qquad ( 23 a )$$

$${v}_{r , 2} ( {x}_{1} , t , {f}_{op} ) = A \cdot cos ( 2 π {f}_{op} \cdot t + θ - \frac{π}{2} ) * {h}_{b , tx} ( {x}_{1} , t , {f}_{op} ) * {h}_{a , rx} ( {x}_{1} , t , 2 {f}_{op} ) * {f}_{s} ( {x}_{1} ) \qquad ( 23 b )$$

$${v}_{r , 3} ( {x}_{1} , t , {f}_{op} ) = A \cdot cos ( 2 π {f}_{op} \cdot t + θ ) * {h}_{a , tx} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 23 c )$$

$${v}_{r , 4} ( {x}_{1} , t , {f}_{op} ) = A \cdot cos ( 2 π {f}_{op} \cdot t + θ - \frac{π}{2} ) * {h}_{b , tx} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 23 d )$$

$${V}_{r , total} ( {x}_{1} , t , {f}_{op} ) = {\sum }_{i = 1}^{4} ( {x}_{1} , t , {f}_{op} ) \qquad ( 24 )$$

$${v}_{r , 2} ( {x}_{1} , t , {f}_{op} ) \neq {v}_{r , n} ( {x}_{1} , t , {f}_{op} ) \qquad ( 25 )$$

$$A \cdot cos ( 2 π {f}_{op} \cdot t + θ - \frac{π}{2} ) * {h}_{b , tx} ( {x}_{1} , t , {f}_{op} ) * {h}_{a , rx} ( {x}_{1} , t , 2 {f}_{op} ) * {f}_{s} ( {x}_{1} ) \neq A \cdot cos ( 2 π {f}_{op} \cdot t + θ ) * {h}_{a , tx} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) ) \qquad ( 26 )$$

The first four equations which represent the four transmits have an additional variable ‘fₒₚ’ added to show that the Fresnel apertures for both transmit and receive are functions of the operational frequency ‘fₒₚ’. The operational frequency on receive is twice the operational frequency on transmit. Of course, the receive frequency may be varied based on where the harmonics are generated and does not have to be twice the transmit frequency. In transmit, two orthogonal apertures are used to produce the ideal phasing for a transmit aperture at ‘fₒₚ’. In receive, two orthogonal apertures are used to produce the ideal phasing for the harmonic frequency which is ‘2fₒₚ’ in this representation. Since the filtered FTHI uses a special receive aperture to focus on the harmonic frequency, the fundamental frequency suppression is better than traditional THI if the same received beamforming filters are applied. It is important to note that the number of transmits for filtered Fresnel THI can be reduced to one if an aperture is used that has both sine and cosine excitations as well as odd and even biases available simultaneously. In this case, the receive aperture is approximately twice the transmit frequency.

### Pulse-Inversion Fresnel Tissue Harmonic Imaging

Eight transmits are required if using pulse-inversion FTHI. This is because the received responses are summed together such that any energy at the fundamental frequency is eliminated and only received signal at the harmonics remains. Additional filtering may be used on the received signal to further isolate the harmonic energy of interest. Pulse-inversion FTHI also has the advantage over standard techniques in that the receive apertures are designed to focus at one frequency. The inverted transmit aperture may be applied using the bias lines or the excitation on the signal line. The eight equations below show that two transmit apertures are required to generate the ideal phasing. Similarly, two receive apertures are required to generate the ideal phasing for each transmit aperture. Therefore, four transmit-receive events are required to generate ideal phasing on both transmit and receive. This doubles to eight transmit-receive events for pulse-inversion FTHI since the inverted transmit also requires four transmit-receive events to generate ideal phasing in both transmit and receive.

$${v}_{r , 1} ( {x}_{1} , t , {f}_{op} ) = A \cdot cos ( 2 π {f}_{op} \cdot t + θ ) * {h}_{a , tx} ( {x}_{1} , t , {f}_{op} ) * {h}_{a , rx} ( {x}_{1} , t , 2 {f}_{op} ) * {f}_{s} ( {x}_{1} ) \qquad ( 27 a )$$

$${v}_{r , 2} ( {x}_{1} , t , {f}_{op} ) = A \cdot cos ( 2 π {f}_{op} \cdot t + θ - \frac{π}{2} ) * {h}_{b , tx} ( {x}_{1} , t , {f}_{op} ) * {h}_{a , rx} ( {x}_{1} , t , 2 {f}_{op} ) * {f}_{s} ( {x}_{1} ) \qquad ( 27 b )$$

$${v}_{r , 3} ( {x}_{1} , t , {f}_{op} ) = A \cdot cos ( 2 π {f}_{op} \cdot t + θ ) * {h}_{a , tx} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 27 c )$$

$${v}_{r , 4} ( {x}_{1} , t , {f}_{op} ) = A \cdot cos ( 2 π {f}_{op} \cdot t + θ - \frac{π}{2} ) * {h}_{b , tx} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 27 d )$$

$${v}_{r , 5} ( {x}_{1} , t , {f}_{op} ) = - A \cdot cos ( 2 π {f}_{op} \cdot t + θ ) * {h}_{a , tx} ( {x}_{1} , t , {f}_{op} ) * {h}_{a , rx} ( {x}_{1} , t , 2 {f}_{op} ) * {f}_{s} ( {x}_{1} ) \qquad ( 27 e )$$

$${v}_{r , 6} ( {x}_{1} , t , {f}_{op} ) = - A \cdot cos ( 2 π {f}_{op} \cdot t + θ - \frac{π}{2} ) * {h}_{b , tx} ( {x}_{1} , t , {f}_{op} ) * {h}_{a , rx} ( {x}_{1} , t , 2 {f}_{op} ) * {f}_{s} ( {x}_{1} ) \qquad ( 27 f )$$

$${v}_{r , 7} ( {x}_{1} , t , {f}_{op} ) = - A \cdot cos ( 2 π {f}_{op} \cdot t + θ ) * {h}_{a , tx} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 27 g )$$

$${v}_{r , 8} ( {x}_{1} , t , {f}_{op} ) = - A \cdot cos ( 2 π {f}_{op} \cdot t + θ - \frac{π}{2} ) * {h}_{{b}_{3} t x} ( {x}_{1} , t , {f}_{op} ) * {h}_{b , rx} ( {x}_{1} , t - \frac{1}{8 \cdot {f}_{op}} , 2 {f}_{op} ) * {f}_{s} ( {x}_{1} ) \qquad ( 27 h )$$

$${V}_{r , total} ( {x}_{1} , t , {f}_{op} ) = {\sum }_{r , n}^{8} ( {x}_{1} , t , {f}_{op} ) \qquad ( 28 )$$

It is important to note that the number of transmits for pulse-inversion Fresnel THI can be reduced to two if an aperture is used that has both sine and cosine excitations as well as odd and even biases available simultaneously. In this case, the receive aperture is approximately twice the transmit frequency and the two transmits are opposites of each other (negative)

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A system for performing ultrasound imaging, the system comprising:
an array of ultrasound transducer elements, each ultrasound element being capable of acoustic transduction when a bias is applied thereto, such that a phase of ultrasound waves emitted therefrom is dependent on a polarity of the bias;
a set of bias conductive paths, each bias conductive path being in electrical communication with a respective bias electrode of an ultrasound element, thereby enabling each ultrasound element to be individually biased;
a set of signal conductive paths, each signal conductive path being configured to deliver a respective signal to a respective subarray of ultrasound elements, thereby enabling the respective signal to be applied to each ultrasound element of the respective subarray of ultrasound elements; and
control and processing circuitry operatively coupled to said set of signal conductive paths and said set of bias conductive paths, said control and processing circuitry comprising at least one processor and associated memory, said memory comprising instructions executable by said processor to perform operations for controlling synthetic transmission of ultrasound energy from said array of ultrasound transducer elements according to a transmit phase aperture, the operations comprising:
performing a first transmit operation comprising:
while applying a first transmit bias aperture to said bias conductive paths, delivering a first set of time-delayed transmit signals to said set of signal conductive paths, each transmit signal of the first set of time-delayed transmit signals being delivered to a respective subarray with a respective coarse transmit subarray delay associated with the transmit phase aperture;
performing a second transmit operation comprising:
while applying a second transmit bias aperture to said bias conductive paths, delivering a second set of time-delayed transmit signals to said signal conductive paths, the second set of time-delayed transmit signals being generated in quadrature relative to the first set of time-delayed transmit signals;
wherein the first transmit bias aperture and the second transmit bias aperture are configured such that when the first transmit operation and the second transmit operation are performed, a fine phase delay associated with the transmit phase aperture is synthetically generated for each transducer element, such that a combination of the per-subarray coarse transmit subarray delays and the per-element fine phase delays synthetically generate or approximate the transmit phase aperture.

2. The system according to claim 1 wherein said control and processing circuitry is further configured such that for at least one subarray, the coarse transmit subarray delay associated with the subarray is a statistical measure generated based on processing a set of per-element time delays, within the subarray that would be needed to generate the transmit phase aperture according to a single transmit operation.

3. system according to claim 1 wherein said control and processing circuitry is further configured such that for at least one subarray, the coarse transmit subarray delay associated with the subarray is determined based on a relative geometrical location of the subarray within the array of ultrasound transducer elements.

4. The system according to claim 1 wherein said control and processing circuitry is configured such that bias levels of the first transmit bias aperture and the second transmit bias aperture are generated according to a discrete set of bias levels, the discrete set of bias levels comprising at least three distinct bias levels.

5. The system according to claim 4 wherein said control and processing circuitry is configured such that the bias levels of the first transmit bias aperture and the bias levels of the second transmit bias aperture are obtained from a lookup table, the lookup table associating, for each phase range of a plurality of phase ranges, a suitable first transmit aperture bias value selected from the discrete set of bias levels and a suitable second transmit aperture bias level selected from the discrete set of bias levels, such that a synthetic transmit aperture generated by compounding of the first transmit operation and the second transmit operation approximates the transmit phase aperture.

6. The system according to claim 1 wherein said control and processing circuitry is further configured such that for at least one subarray, the first transmit operation is performed as a set of first synthetic transmit operations and the second transmit operation is performed as a corresponding set of second synthetic transmit operations, and wherein the set of first synthetic transmit operations and the set of second synthetic transmit operations are configured to reduce or avoid phase wrapping within the subarray.

7. The system according to claim 6 wherein said control and processing circuitry is further configured such that when performing a given first synthetic transmit operation associated with the first transmit operation, a respective subaperture of the elements of the subarray is biased according to the first transmit bias aperture with a remainder of the elements of the subarray being unbiased, and wherein the given first synthetic transmit operation has a corresponding second synthetic transmit operation associated with the second transmit operation, in which the subaperture of the electrodes of the subarray is biased according to the second transmit bias aperture with the remainder of the elements of the subarray being unbiased, and wherein the given first synthetic transmit operation and the corresponding second synthetic transmit operation are performed using a coarse transmit subaperture delay selected to reduce or avoid phase wrapping within the subaperture of elements of the subarray.

8. The system according to claim 7 wherein said control and processing circuitry is further configured such that the coarse transmit subaperture delay is a statistical measure generated based on processing a set of per-element time delays, within the subaperture of the subarray, that would be needed to generate the transmit phase aperture according to a single transmit operation.

9. The system according to claim 6 wherein said control and processing circuitry is further configured such that the number of synthetic transmit operations associated with a given subarray is dependent on a focal location associated with the transmit phase aperture.

10. The system according to claim 6 wherein said control and processing circuitry is further configured such that the number of synthetic transmit operations associated with a given subarray is selected to minimize a variation in a signal-to-noise ratio among synthetic transmit operations associated with the given subarray.

11. The system according to claim 1 wherein two or more of the subarrays have different sizes.

12. The system according to claim 11 wherein a central subarray has a larger size than a peripheral subarray.

13. The system according to claim 1 wherein at least on subarray is sufficiently small to avoid phase wrapping within a pre-selected steering range.

14. The system according to claim 1 wherein said control and processing circuitry is configured such that the transmit phase aperture associated with a real focus, such that the first transmit operation and the second transmit operation synthetically focus ultrasound energy at the real focus.

15. The system according to claim 14 wherein the real focus is a first real focus, and wherein said control and processing circuitry is configured such that an additional first transmit operation and an additional second transmit operation are performed to synthetically focus ultrasound energy at a second real focus residing proximal to the first real focus, wherein the additional first transmit operation is performed using the first transmit bias aperture and the additional second transmit operation is performed using the second transmit bias aperture, such that the second real focus is obtained by modifying the coarse transmit subarray delays applied to the transmit signals without modifying the first transmit bias aperture and the second transmit bias aperture.

16. The system according to claim 14 wherein said control and processing circuitry is configured to perform additional first and second transmit operations to synthetically focus ultrasound energy at a plurality of focal locations within a selected sector by modifying the coarse transmit subarray delays applied to the transmit signals without modifying the first transmit bias aperture and the second transmit bias aperture.

17. The system according to claim 1 wherein said control and processing circuitry is configured such that the transmit phase aperture is associated with a virtual focus, such that the first transmit operation and the second transmit operation synthetically generate ultrasound energy according to the virtual focus.

18. The system according to claim 1 wherein said control and processing circuitry is further configured to perform additional operations for synthetically receiving ultrasound energy according to a receive phase aperture, comprising:
in response to the first transmit operation, performing a first receive operation by receiving a first set of receive signals while applying a first receive bias aperture;
in response to the second transmit operation, performing a second receive operation by receiving a second set of receive signals while applying the first receive bias aperture;
performing a third transmit operation by repeating the first transmit operation, and in response to the third transmit operation, performing a third receive operation by receiving a third set of receive signals while applying a second receive bias aperture and applying a quarter-wave time delay to the third set of receive signals;
performing a fourth transmit operation by repeating the second transmit operation, and in response to the fourth transmit operation, performing a fourth receive operation by receiving a fourth set of receive signals while applying the second receive bias aperture and applying a quarter-wave time delay to the fourth set of receive signals;
wherein the first receive bias aperture and the second receive bias aperture are configured to synthetically generate a fine phase delay associated with the receive phase aperture;
beamforming each of the first set of receive signals, the second set of receive signals, the third set of receive signals and the fourth set of receive signals, according to coarse receive subaperture delays associated with the receive phase aperture, and summing the resulting first beamformed receive signal, second beamformed receive signal, third beamformed receive signal, a fourth beamformed receive signal to obtain a final receive beamformed signal.

19. The system according to claim 18 wherein said control and processing circuitry is configured such that bias levels of the first receive bias aperture and the second receive bias aperture are generated according to a discrete set of bias levels, the discrete set of bias levels comprising at least three distinct bias levels.

20. The system according to claim 19 wherein said control and processing circuitry is configured such that bias levels of the first receive bias aperture and the bias levels of the second receive bias aperture are obtained from a lookup table, the lookup table associating, for each phase range of a plurality of phase ranges, a suitable first receive aperture bias value selected from the discrete set of bias levels and a suitable second receive aperture bias level selected from the discrete set of bias levels, such that a synthetic receive aperture generated by the compounding of the receive operations approximates the receive phase aperture.

21. The system according to claim 18 wherein said control and processing circuitry is configured such that the set of synthetic transmit and receive operations are performed in a sequence that minimizes switching between the bias apertures.

22. The system according to claim 18 wherein the first receive bias aperture is the same as the first transmit bias aperture and the second receive bias aperture is the same as the second transmit bias aperture.

23. The system according to claim 18 wherein the first transmit operation, the second transmit operation, the third transmit operation, and the fourth transmit operation are a first set of synthetic transmit operations and wherein the first receive operation, the second receive operation, the third receive operation, and the fourth receive operation are a first set of synthetic receive operations, and wherein said control and processing circuitry is configured to perform at least one additional set of synthetic transmit operations and at least one additional set of synthetic receive operations, wherein each set of synthetic transmit operations is configured to synthetically generate an ultrasound field that approximates a plane wave, and wherein the plane waves associated with the sets of synthetic transmit operations spatially overlap within a region;
wherein each set of synthetic receive operations is configured to synthetically focus ultrasound energy from a different location within the region.

24. The system according to claim 23 wherein said control and processing circuitry is configured such that each set of synthetic receive operations synthetically focuses ultrasound energy from a different location by modifying the coarse receive subarray delays in the absence of modifying the first receive bias aperture and the second receive bias aperture.

25. The system according to claim 23 wherein said control and processing circuitry is configured such that each set of synthetic receive operations synthetically focuses ultrasound energy from a different location, at least in part, by modifying the coarse receive subarray delays, and wherein at least two different pairs of the first receive bias aperture and the second receive bias aperture are employed when performing the sets of synthetic receive operations.

26. The system according to claim 1 wherein the array of ultrasound transducer elements comprises an electrostrictive material.

27. The system according to claim 1 wherein the array of ultrasound transducer elements is formed from an array of capacitive micromachined ultrasound transducer elements.

28. A method of performing ultrasound imaging, the method comprising:
providing an ultrasound device comprising:
an array of ultrasound transducer elements, each ultrasound element being capable of acoustic transduction when a bias is applied thereto, such that a phase of ultrasound waves emitted therefrom is dependent on a polarity of the bias;
a set of bias conductive paths, each bias conductive path being in electrical communication with a respective bias electrode of an ultrasound element, thereby enabling each ultrasound element to be individually biased; and
a set of signal conductive paths, each signal conductive path being configured to deliver a respective signal to a respective subarray of ultrasound elements, thereby enabling the respective signal to be applied to each ultrasound element of the respective subarray of ultrasound elements; and
performing a first transmit operation comprising:
while applying a first transmit bias aperture to said bias conductive paths, delivering a first set of time-delayed transmit signals to said set of signal conductive paths, each transmit signal of the first set of time-delayed transmit signals being delivered to a respective subarray with a respective coarse transmit subarray delay associated with a transmit phase aperture;
performing a second transmit operation comprising:
while applying a second transmit bias aperture to said bias conductive paths, delivering a second set of time-delayed transmit signals to said signal conductive paths, the second set of time-delayed transmit signals being generated in quadrature relative to the first set of time-delayed transmit signals;
wherein the first transmit bias aperture and the second transmit bias aperture are configured such that when the first transmit operation and the second transmit operation are performed, a fine phase delay associated with the transmit phase aperture is synthetically generated for each transducer element, such that a combination of the per-subarray coarse transmit subarray delays and the per-element fine phase delays synthetically generate or approximate the transmit phase aperture.

29. A system for delivering ultrasound energy, the system comprising:
an array of ultrasound transducer elements, each ultrasound element comprising a first sub-element and a second sub-element residing adjacent to one another, each sub-element being capable of acoustic transduction when a bias is applied thereto, such that a phase of ultrasound waves emitted therefrom is dependent on a polarity of the bias;
a first set of bias conductive paths, each first bias conductive path being in electrical communication with a respective bias electrode of a first sub-element, thereby enabling each first sub-element to be individually biased;
a second set of bias conductive paths, each second bias conductive path being in electrical communication with a respective bias electrode of a second sub-element, thereby enabling each second sub-element to be individually biased;
a first set of signal conductive paths, each first signal conductive path being configured to deliver a respective signal to a respective set of first sub-elements of a subarray of ultrasound elements, thereby enabling the respective signal to be applied to each first sub-element of the respective subarray of ultrasound elements;
a second set of signal conductive paths, each second signal conductive path being configured to deliver a respective signal to a respective set of second sub-elements of a subarray of ultrasound elements, thereby enabling the respective signal to be applied to each second sub-element of the respective subarray of ultrasound elements; and
control and processing circuitry operatively coupled to said set of signal conductive paths, said first set of bias conductive paths and said second set of bias conductive paths, said control and processing circuitry comprising at least one processor and associated memory, said memory comprising instructions executable by said processor to perform operations for controlling transmission of ultrasound energy from said array of ultrasound transducer elements according to a transmit phase aperture, the operations comprising:
simultaneously performing a first transmit operation and a second transmit operation;
the first transmit operation comprising:
while applying a first transmit bias aperture to said first set of bias conductive paths, delivering a first set of time-delayed transmit signals to said first set of signal conductive paths, each transmit signal of the first set of time-delayed transmit signals being delivered to a respective subarray with a respective coarse transmit subarray delay associated with the transmit phase aperture;
the second transmit operation comprising:
while applying a second transmit bias aperture to said second bias conductive paths, delivering a second set of time-delayed transmit signals to said second set of signal conductive paths, the second set of time-delayed transmit signals being generated in quadrature relative to the first set of time-delayed transmit signals;
the first transmit bias aperture and the second transmit bias aperture being configured such that when the first transmit operation and the second transmit operation are simultaneously performed, each pair of first and second sub-elements generates a net fine phase delay associated with the transmit phase aperture, such that a combination of the per-subarray coarse transmit subarray delays and the per-sub-element fine phase delays generate or approximate the transmit phase aperture.

30. The system according to claim 29 wherein said control and processing circuitry is further configured such that for at least one subarray, the coarse transmit subarray delay associated with the subarray is a statistical measure generated based on processing a set of per-element time delays, within the subarray that would be needed to generate the transmit phase aperture according to a single transmit operation.

31. The system according to claim 29 wherein said control and processing circuitry is further configured such that for at least one subarray, the coarse transmit subarray delay associated with the subarray is determined based on a relative geometrical location of the subarray within the array of ultrasound transducer elements.

32. The system according to claim 29 wherein said control and processing circuitry is configured such that bias levels of the first transmit bias aperture and the second transmit bias aperture are generated according to a discrete set of bias levels, the discrete set of bias levels comprising at least three distinct bias levels.

33. The system according to claim 32 wherein said control and processing circuitry is configured such that the bias levels of the first transmit bias aperture and the bias levels of the second transmit bias aperture are obtained from a lookup table, the lookup table associating, for each phase range of a plurality of phase ranges, a suitable first transmit aperture bias value selected from the discrete set of bias levels and a suitable second transmit aperture bias level selected from the discrete set of bias levels, such that the first transmit operation and the second transmit operation approximates the transmit phase aperture.

34. The system according to claim 29 wherein two or more of the subarrays have different sizes.

35. The system according to claim 34 wherein a central subarray has a larger size than a peripheral subarray.

36. The system according to claim 29 wherein at least on subarray is sufficiently small to avoid phase wrapping within a pre-selected steering range.

37. The system according to claim 29 wherein said control and processing circuitry is configured such that the transmit phase aperture associated with a real focus, such that the first transmit operation and the second transmit operation focus ultrasound energy at the real focus.

38. The system according to claim 37 wherein the real focus is a first real focus, and wherein said control and processing circuitry is configured such that an additional first transmit operation and an additional second transmit operation are performed to focus ultrasound energy at a second real focus residing proximal to the first real focus, wherein the additional first transmit operation is performed using the first transmit bias aperture and the additional second transmit operation is performed using the second transmit bias aperture, such that the second real focus is obtained by modifying the coarse transmit subarray delays applied to the transmit signals without modifying the first transmit bias aperture and the second transmit bias aperture.

39. The system according to claim 37 wherein said control and processing circuitry is configured to perform additional first and second transmit operations to focus ultrasound energy at a plurality of focal locations within a selected sector by modifying the coarse transmit subarray delays applied to the transmit signals without modifying the first transmit bias aperture and the second transmit bias aperture.

40. The system according to claim 29 wherein said control and processing circuitry is configured such that the transmit phase aperture is associated with a virtual focus, such that the first transmit operation and the second transmit operation generate ultrasound energy according to the virtual focus.

41. The system according to claim 29 wherein said control and processing circuitry is further configured to perform additional operations for receiving ultrasound energy according to a receive phase aperture, comprising:
in response to the first transmit operation and the second transmit operation:
performing a first receive operation by receiving a first set of receive signals from said first set of signal conductive paths while applying a first receive bias aperture to said first set of bias conductive paths; and
performing a second receive operation by receiving a second set of receive signals from said second set of signal conductive paths while applying a second receive bias aperture and applying a quarter-wave time delay to the second set of receive signals;
simultaneously performing a third transmit operation and a fourth transmit operation by repeating the first transmit operation and the second transmit operation;
in response to the third transmit operation and the fourth transmit operation:
performing a third receive operation by receiving a third set of receive signals from said first set of signal conductive paths while applying the second receive bias aperture to said first set of bias conductive paths and applying a quarter-wave time delay to the second set of receive signals; and
performing a fourth receive operation by receiving a fourth set of receive signals from said second set of signal conductive paths while applying the first receive bias aperture;
wherein the first receive bias aperture and the second receive bias aperture are configured to synthetically generate a fine phase delay associated with the receive phase aperture;
beamforming each of the first set of receive signals, the second set of receive signals, the third set of receive signals and the fourth set of receive signals, according to coarse receive subaperture delays associated with the receive phase aperture, and summing the resulting first beamformed receive signal, second beamformed receive signal, third beamformed receive signal, a fourth beamformed receive signal to obtain a final receive beamformed signal.

42. The system according to claim 41 wherein said control and processing circuitry is configured such that bias levels of the first receive bias aperture and the second receive bias aperture are generated according to a discrete set of bias levels, the discrete set of bias levels comprising at least three distinct bias levels.

43. The system according to claim 42 wherein said control and processing circuitry is configured such that bias levels of the first receive bias aperture and the bias levels of the second receive bias aperture are obtained from a lookup table, the lookup table associating, for each phase range of a plurality of phase ranges, a suitable first receive aperture bias value selected from the discrete set of bias levels and a suitable second receive aperture bias level selected from the discrete set of bias levels, such that the receive operations approximate the receive phase aperture.

44. The system according to claim 41 wherein the first receive bias aperture is the same as the first transmit bias aperture and the second receive bias aperture is the same as the second transmit bias aperture.

45. The system according to claim 41 wherein the first transmit operation, the second transmit operation, the third transmit operation, and the fourth transmit operation are a first set of transmit operations and wherein the first receive operation, the second receive operation, the third receive operation, and the fourth receive operation are a first set of receive operations, and wherein said control and processing circuitry is configured to perform at least one additional set of transmit operations and at least one additional set of receive operations, wherein each set of transmit operations is configured to generate an ultrasound field that approximates a plane wave, and wherein the plane waves associated with the sets of synthetic transmit operations spatially overlap within a region;
wherein each set of receive operations is configured to focus ultrasound energy from a different location within the region.

46. The system according to claim 45 wherein said control and processing circuitry is configured such that each set of receive operations focuses ultrasound energy from a different location by modifying the coarse receive subarray delays in the absence of modifying the first receive bias aperture and the second receive bias aperture.

47. The system according to claim 45 wherein said control and processing circuitry is configured such that each set of receive operations focuses ultrasound energy from a different location, at least in part, by modifying the coarse receive subarray delays, and wherein at least two different pairs of the first receive bias aperture and the second receive bias aperture are employed when performing the sets of receive operations.

48. The system according to claim 29 wherein the array of ultrasound transducer elements comprises an electrostrictive material.

49. The system according to claim 29 wherein the array of ultrasound transducer elements is formed from an array of capacitive micromachined ultrasound transducer elements.

50. A method for delivering ultrasound energy, the system comprising:
providing an ultrasound device comprising:
an array of ultrasound transducer elements, each ultrasound element comprising a first sub-element and a second sub-element residing adjacent to one another, each sub-element being capable of acoustic transduction when a bias is applied thereto, such that a phase of ultrasound waves emitted therefrom is dependent on a polarity of the bias;
a first set of bias conductive paths, each first bias conductive path being in electrical communication with a respective bias electrode of a first sub-element, thereby enabling each first sub-element to be individually biased;
a second set of bias conductive paths, each second bias conductive path being in electrical communication with a respective bias electrode of a second sub-element, thereby enabling each second sub-element to be individually biased;
a first set of signal conductive paths, each first signal conductive path being configured to deliver a respective signal to a respective set of first sub-elements of a subarray of ultrasound elements, thereby enabling the respective signal to be applied to each first sub-element of the respective subarray of ultrasound elements; and
a second set of signal conductive paths, each second signal conductive path being configured to deliver a respective signal to a respective set of second sub-elements of a subarray of ultrasound elements, thereby enabling the respective signal to be applied to each second sub-element of the respective subarray of ultrasound elements; and
simultaneously performing a first transmit operation and a second transmit operation;
the first transmit operation comprising:
while applying a first transmit bias aperture to said first set of bias conductive paths, delivering a first set of time-delayed transmit signals to said first set of signal conductive paths, each transmit signal of the first set of time-delayed transmit signals being delivered to a respective subarray with a respective coarse transmit subarray delay associated with a transmit phase aperture;
the second transmit operation comprising:
while applying a second transmit bias aperture to said second bias conductive paths, delivering a second set of time-delayed transmit signals to said second set of signal conductive paths, the second set of time-delayed transmit signals being generated in quadrature relative to the first set of time-delayed transmit signals;
the first transmit bias aperture and the second transmit bias aperture being configured such that when the first transmit operation and the second transmit operation are simultaneously performed, each pair of first and second sub-elements generates a net fine phase delay associated with the transmit phase aperture, such that a combination of the per-subarray coarse transmit subarray delays and the per-sub-element fine phase delays generate or approximate the transmit phase aperture.
